(19) United States
(12) Patent Application Publication
(10) Pub. No.: US 2014/0336181 A1
(43) Pub. Date: Nov. 13, 2014

(54) Use of polymorphisms for identifying individuals at risk of developing autism

(71) Applicant: Amy Yasko, Bethel, ME (US)

(72) Inventor: Amy Yasko, Bethel, ME (US)

(21) Appl. No.: 14/092,185

(22) Filed: Nov. 27, 2013

Related U.S. Application Data
(63) Continuation-in-part of application No. 13/480,261, filed on May 24, 2012.

(51) Int. Cl. C12Q 1/68

(52) U.S. Cl. CPC C12Q 1/6883; C12Q 2600/118
USPC 514/217; 435/6.11

(57) ABSTRACT

The present invention relates to nucleic-acid based diagnostics and the use of such diagnostics for the diagnosis of developmental disorders. Novel methods of assessing individuals for the risk of developing autism through the identification of mutations of the Cystic Fibrosis Transmembrane Conductance Regulator (CFTR) gene alone or in combination with other genes associated with methylation pathways are identified. Methods of identifying those individuals that are at increased and/or decreased risk for developing autism are provided.

### PRIORITY CLAIM

In accordance with 37 C.F.R. 1.76, a claim of priority is included in an Application Data Sheet filed concurrently herewith. Accordingly, the present invention claims priority as a continuation-in-part of U.S. patent application Ser. No. 13/480,261, entitled “USE OF POLYMORPHISMS FOR IDENTIFYING INDIVIDUALS AT RISK OF DEVELOPING AUTISM”, filed May 24, 2012. The contents of the above referenced application is incorporated herein by reference in its entirety.

### FIELD OF THE INVENTION

The present invention relates to the field of diagnostics for developmental disorders and to the use of nucleic-acid based diagnostics. More particularly, the present invention relates to the use of single nucleotide polymorphisms (SNPs) for identifying individuals that have increased and/or decreased risks for developing autism and related pathologies.

### BACKGROUND OF THE INVENTION

Autism is one of the types of complex neurodevelopment disorders known as Autism Spectrum Disabilities (ASD). Autism is generally characterized by social impairments, difficulty in communicating and stereotypical behavioral patterns, such as failure to make eye contact or engaging in a repetitive movement. However, individuals with autism are affected in different ways, and the severity and exact nature of the symptoms can differ dramatically among individuals. Autism can occur in all racial and economic groups. A 2009 report by the CDC indicated that the prevalence of autism in the United States had risen to 1 in 88 (http://www.cdc.gov/ncbddd/autism/data.html), with the prevalence for boys being 1 in 70. Understanding the predisposing factors that are vital to the development of the disorder is a critical step in preventing future cases. While the exact cause of Autism is unknown, it is believed to be a combination of genetic and/or environmental factors. Moreover, evidence suggests that individuals with autism may suffer from several imbalances which impact the proper functioning of organ systems, neurotransmitters, and many interactive biochemical processes in the body.

The costs associated with treating autism are estimated to reach 200 billion dollars over the next 10-20 years. For those having to care for a family member with autism, the cost per family for raising a child to the age of 22 is estimated to be over one million dollars. Costs over an individual's lifetime can reach close to four million dollars. Understanding the predisposing factors that play a role in developing Autism is vital. Diagnosing autism can be difficult as there is not a current direct method of detection, such as a blood test.

In any given population of individuals, there exist variants (alleles) of a given gene. Alleles can differ from one another in different ways, such as single base substitutions, insertion/deletion, or short tandem repeats. Single nucleotide polymorphisms (SNPs) are single base pair differences, and several can be present in a single gene. High-throughput genotyping methods using high-density nucleic acid arrays and other methods can accurately genotype (i.e., determine the nucleotide(s) present) hundreds or thousands of SNPs in a genetic sample in parallel to provide an unequivocal molecular fingerprint of the genetic sample. Accordingly, methods for screening and diagnosing disease states using SNPs is known in the art, see for example U.S. Pat. No. 8,124,345 (method of determining whether a human has a predisposition to cystic fibrosis by determining the presence of Cystic Fibrosis Transmembrane Conductance Regulator (CFTR) mutations, U.S. Patent Application Publication Number 2011/0046094 (method of assessing whether an individual is at risk for developing Lupus by detecting one or more SNPs), U.S. Patent Application Publication Number 2011/0039918 (method of screening for and identifying susceptibility to or detecting risk in developing inflammatory disease by detecting one or more SNP identified in a gene associated with inflammatory disease, U.S. Patent Application Publication Number 2008/0286796 (method of determining if an individual has an altered risk for autoinflammatory disease), U.S. Patent Application Publication Number 2007/0072821 (method of identifying individuals having altered risk for developing acute coronary event by detecting one or more SNPs. Use of SNP detection methods may present an important mechanism for determining individuals at risk of developing and/or diagnosing autism as there is no assay-based medical test currently available.

Autism typically appears within the first 3 years of life. Despite the early onset of the disease, no known nucleic acid based test has been developed in order to determine risk and/or diagnosis of autism and related disorders. Current diagnosis for autism includes assessing an individual's behavior and development status through administration of specifically designed behavioral evaluations. The evaluation is used to determine if a person has autism and to what degree. The disadvantage of this testing method is that it is not predictive for assessing the risk for developing the disorder. Even if a person is found to have autism, there currently exists no known magic bullet that cures a person from the disease. While a cure for autism has not been found, treatment options for those suffering the disease are available. Given the complexity of the disease, treatment options include the use of one or more of behavior and communication therapies, educational therapies, nutritional therapies, or use of medication. Early intervention has been shown to help alleviate or dramatically reduce the level of severity of some of the patients suffering from this disease. In fact, early intensive behavioral intervention has been shown to produce marked changes in the skill deficits and problem behavior associated with the disorder.

Therefore, there is a need for novel diagnostic tests for identifying those individuals with an increased risk of developing autism and for diagnostic purposes. The use of a nucleic acid type test, such as a SNP based testing panel and method of using such a panel, which provides a non-invasive mechanism to determine if an individual is susceptible to or suffers from autism may provide physicians and families a more accurate assessment tool. Such testing may provide an early detection system and aid in designing treatment options. For those individuals identified as having an increased risk, treatment plans can be developed at an early stage so as to minimize, delay, reverse, reduce the severity, or prevent the onset of the disorder.

### SUMMARY OF THE INVENTION

The present invention relates to nucleic-acid based diagnostics and to the use of such diagnostics for the identification of developmental disorders, such as autism and related pathologies. Single nucleotide polymorphisms (SNPs) in the human genome were found in patients clinically diagnosed with autism. While the SNPs disclosed herein can be used as targets in the development of therapeutic agents, the present invention is most useful in providing a panel containing one or more of the SNPs for identifying those individuals that have increased and/or decreased risks for developing autism in order to develop treatment plans as early as possible. The present invention includes methods, assays, and kits for detecting the presence of one or more of the SNPs identified, thereby providing a genetic signature indicative of the risk of developing or diagnosing autism.

Accordingly, it is an objective of the present invention to provide diagnostics for developmental disorders.

It is a further objective of the present invention to provide nucleic acid based diagnostics for developmental disorders.

It is yet another objective of the present invention to provide diagnostic tests using single nucleotide polymorphisms (SNPs) for identifying individuals that have increased and/or decreased risks for developing developmental disorders.

It is a still further objective of the present invention to provide diagnostic tests using single nucleotide polymorphisms (SNPs) for identifying individuals that have increased and/or decreased risks for developing autism and related pathologies.

It is a further objective of the present invention to provide methods for identifying individuals having increased and/or decreased risks for developing autism and related pathologies.

It is yet another objective of the present invention to provide methods for identifying individuals having increased and/or decreased risks for developing autism and related pathologies using single nucleotide polymorphisms (SNPs).

It is a still further objective of the present invention to provide a kit useful for identifying individuals having increased and/or decreased risks for developing autism and related pathologies.

It is a further objective of the present invention to provide a kit using a plurality of single nucleotide polymorphisms (SNPs) for identifying individuals having increased and/or decreased risks for developing autism and related pathologies.

It is yet another objective of the present invention to provide a method of identifying individuals having increased and/or decreased risks for developing autism and related pathologies by identifying one or more single nucleotide polymorphisms (SNPs) associated with the CFTR gene.

It is a still further objective of the present invention to provide a method of identifying individuals having increased and/or decreased risks for developing autism and related pathologies by identifying one or more single nucleotide polymorphisms (SNPs) associated with the CFTR gene in combination with identifying one or more single nucleotide polymorphisms (SNPs) associated with one or more genes involved in the methylation pathway.

It is a further objective of the present invention to provide a kit having a plurality of single nucleotide polymorphisms (SNPs) associated with the CFTR gene for identifying individuals having increased and/or decreased risks for developing autism and related pathologies.

It is yet another objective of the present invention to provide a kit for identifying one or more single nucleotide polymorphisms (SNPs) associated with the one or more methylation pathway genes for identifying individuals having increased and/or decreased risks for developing autism and related pathologies.

It is a still further objective of the present invention to provide a kit for identifying one or more single nucleotide polymorphisms (SNPs) associated with the CFRT gene in combination with one or more single nucleotide polymorphisms (SNPs) associated with one or more methylation pathway genes for identifying individuals having increased and/or decreased risks for developing autism and related pathologies.

It is a further objective of the present invention to provide a method of diagnosing autism in an individual.

It is yet another objective of the present invention to provide a method of diagnosing autism or susceptibility to autism in an individual which identifies the presence of one or more mutations in the CFTR gene.

It is a still further objective of the present invention to provide a method of diagnosing autism in an individual which identifies the presence of one or more mutations in the CFTR gene and one or more genes in the methylation pathway.

It is a further objective of the present invention to provide a set of SNPs comprising a genetic signature indicative of autism.

It is yet another objective of the present invention to provide a set of SNPs comprising a genetic signature indicative of the risk of developing autism.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1A provides identification for each CFTR related SNP in accordance with the present invention;

FIG. 1B provides the flanking sequenced of the SNPs illustrated in FIG. 1A;

FIG. 1C provides location on the chromosome for the SNPs illustrated in FIG. 1A;

FIG. 2A provides identification for each methylation pathway related SNP in accordance with the present invention;

FIG. 2B provides the flanking sequenced of the SNPs illustrated in FIG. 2A;

FIG. 2C provides location on the chromosome for the SNPs illustrated in FIG. 2A;

FIG. 3 is a diagram illustrating the biological processes associated with the methylation cycle;

FIG. 4A is a bar graph illustrating a clustering effect in which the majority of tested individuals observed as having mutations in CFTR genes were observed to have a plurality of mutations;

FIG. 4B is a bar graph a clustering effect in which the majority of individuals who were observed as having CFTR and methylation mutations were observed to have a plurality of mutations;

FIG. 5 illustrates the percentage of individuals in a test population tested for CFTR and methylation pathway mutations, which contained dual copies of mutations, single mutation, or no mutations.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

Unless defined otherwise, all technical and scientific terms used herein have the same meaning as commonly understood by one of ordinary skill in the art to which the invention pertains.

Probes and primers may be designed to hybridize to either strand, and SNP genotyping methods disclosed herein may generally target either strand. Primers, oligonucleotides, and polynucleotides used or contemplated within the present invention can be generated using standard techniques known in the art.

As used herein, “autism” is defined as one of five disorders, Autistic Disorder, coming under the umbrella of Pervasive Developmental Disorders (PDD), a category of neurological disorders characterized by severe and pervasive impairment in several areas of development, including social interaction and communications skills (DSM-IV-TR). The disease is a complex developmental disability that interferes with, inter alia, the normal development of the brain in the areas of social interaction and communication skills which is the result of a neurological disorder which affects the functioning of the brain. Autistic children and adults have difficulties in verbal and non-verbal communication, social interactions, and leisure or play activities. Specific diagnostic criteria for Autism can be found in the Diagnostic & Statistical Manual of Mental Disorders (DSM-IV-TR) as distributed by the American Psychiatric Association (APA). Although autism is defined by a certain set of behaviors, it is a spectrum disorder such that symptoms and characteristics can be present in a wide variety of combinations, from mild to severe. The term used herein further encompasses clinically diagnosed individuals, pre-clinically diagnosed individuals, such as those individuals that contain one or more of the symptoms but have not been fully diagnosed as Autistic (preclinical), or those individuals that will develop the disease eventually. Individuals having autism may include, but are not limited to, the following traits: Insistence on sameness or resistance to change; Difficulty in expressing needs; (i.e. uses gestures or pointing instead of words); Repeating words or phrases in place of normal, responsive language; Laughing, crying, showing distress for reasons not apparent to others; Prefers to be alone or aloof manner; Tantrums; Difficulty in mixing with others; May not want to cuddle or be cuddled; Little or no eye contact; Unresponsive to normal teaching methods; Sustained odd play; Spins objects; Inappropriate attachments to objects; Apparent over-sensitivity or under-sensitivity to pain; No real fears of danger; Noticeable physical over-activity or extreme under-activity; Uneven gross/fine motor skills; and/or Not responsive to verbal cues (i.e. acts as if deaf although hearing tests in normal range).

As used herein “autism related pathologies” refers to any of the other disorders under PDD including Asperger's Disorder, Childhood Disintegrative Disorder (CDD), Rett's Disorder, and PDD-Not Otherwise Specified (PDD-NOS).

As used herein, the term “SNP” is used to define single nucleotide polymorphisms which are single base substitutions or variations involving A, T, C, or G, as well as SNP variations including insertion/deletion variations, microsatellite or short tandem repeats, named variations (insertion/deletion variation of named repetitive elements, mixed variations (cluster contains submissions from 2 or more allelic classes), or multi-nucleotide polymorphisms (variations that are multi-based variations of a single, common length, such as ACG/TTC). The use of SNP may also be used to define any protein changes as a result of base substitutions or variations.

As used herein, an “allele” may refer to a nucleotide at a SNP position (wherein at least two alternative nucleotides are present in the population at the SNP position, in accordance with the inherent definition of a SNP) or may refer to an amino acid residue that is encoded by the codon which contains the SNP position (where the alternative nucleotides that are present in the population at the SNP position form alternative codons that encode different amino acid residues). An “allele” may also be referred to herein as a “variant”. Also, an amino acid residue that is encoded by a codon containing a particular SNP may simply be referred to as being encoded by the SNP.

As used herein, a “polynucleotide” or “nucleic acid” refers to polymers of nucleotides of any length. The nucleotides can be DNA, RNA, deoxribonucleotides, ribonucleotides, modified nucleotides or bases and/or their analogs.

As used herein, an “oligonucleotide” refers to short single stranded polynucleotides that are preferably between 7 and 250 nucleotides in length. Oligonucleotides may be made synthetically using known techniques in the art.

As used herein, “variation” means any change in a nucleotide sequence relative to a reference sequence such as wild type sequence, including corresponding changes to the compliment nucleotide sequence, or amino acid variation.

As used herein, the term “at risk” refers to those individuals that have an increased risk or decreased risk of having or developing autism or have increased risk or decreased risk of developing mild to severe symptoms and characteristics of the disease.

As used herein, the term “predisposition” or “predisposed” refers to those individuals that are susceptible to developing autism, or susceptible to developing symptoms and characteristics in a wide variety of combinations, from mild to severe, whether as a result of physiological or structural factors, i.e. abnormalities of the brain structure or function, hereditary, i.e. unstable genes that alter brain development, or environmental factors, such as viral or bacterial infections, metabolic imbalances, or exposure to environmental chemicals.

Those skilled in the art will readily recognize that nucleic acid molecules may be double-stranded molecules and that reference to a particular site on one strand refers, as well, to the corresponding site on a complementary strand. In defining a SNP position, SNP allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand (reverse complimentary bases) of the nucleic acid molecule. Thus, reference may be made to either strand in order to refer to a particular SNP position, SNP allele, or nucleotide sequence.

The present invention provides SNPs associated with developmental disorders, such as autism and related pathologies. The present invention further includes nucleic acid molecules containing these SNPs and methods of detection of these SNPs are disclosed herein, uses of these SNPs for the development of reagents, and assays or kits that utilize these reagents. The SNPs disclosed herein are useful for diagnosing, screening, and evaluating predisposition to autism and related pathologies in humans.

The present invention provides a novel method of assessing individuals for the risk of developing autism through the use of SNPs previously known in the art but not known to be associated with autism. Accordingly, the present invention provides for novel compositions and methods of using, as well as novel methods of using already known, but unassociated SNPs, in methods relating to the identification and evaluation of individuals likely to have or develop autism. One or more SNP alleles of the present invention can be associated with an increased risk of having or developing autism. Similarly, lack of one or more of particular SNPs can be associated with a decreased risk of having or developing autism.

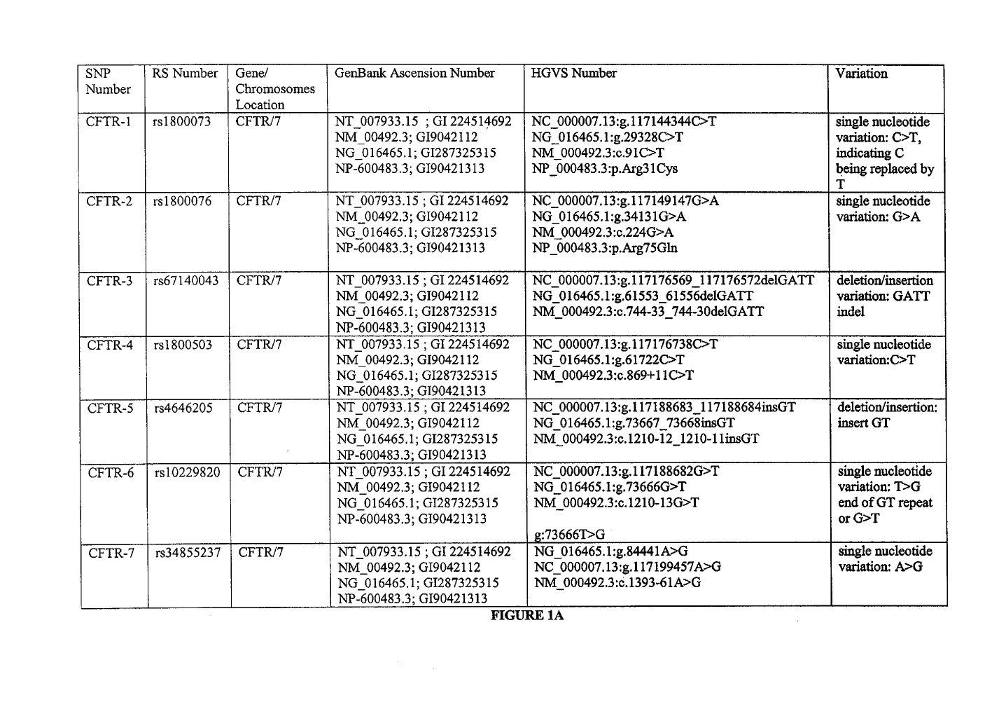

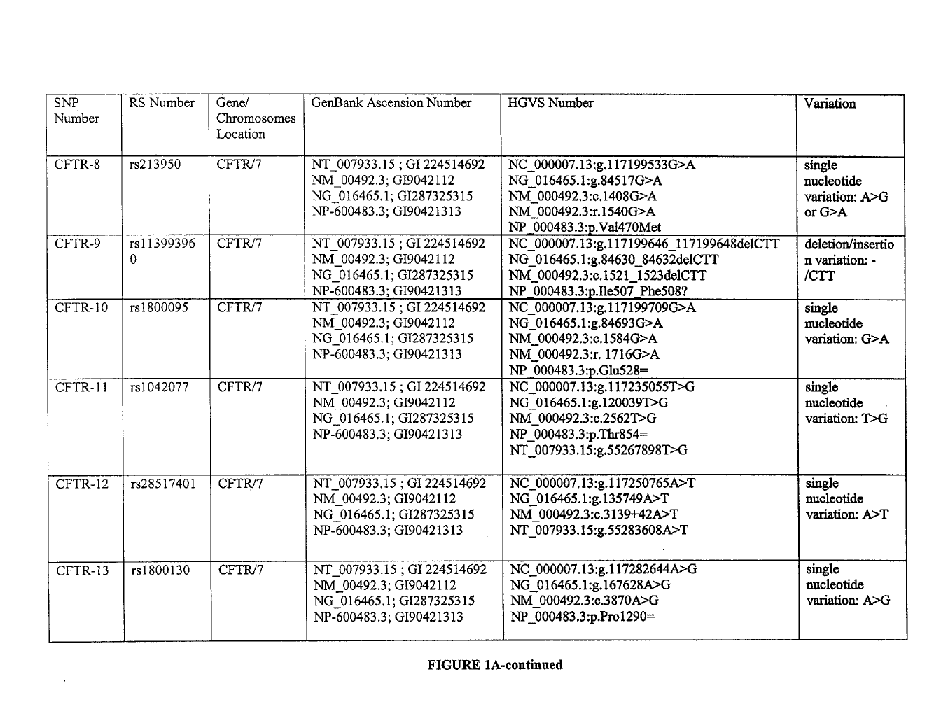

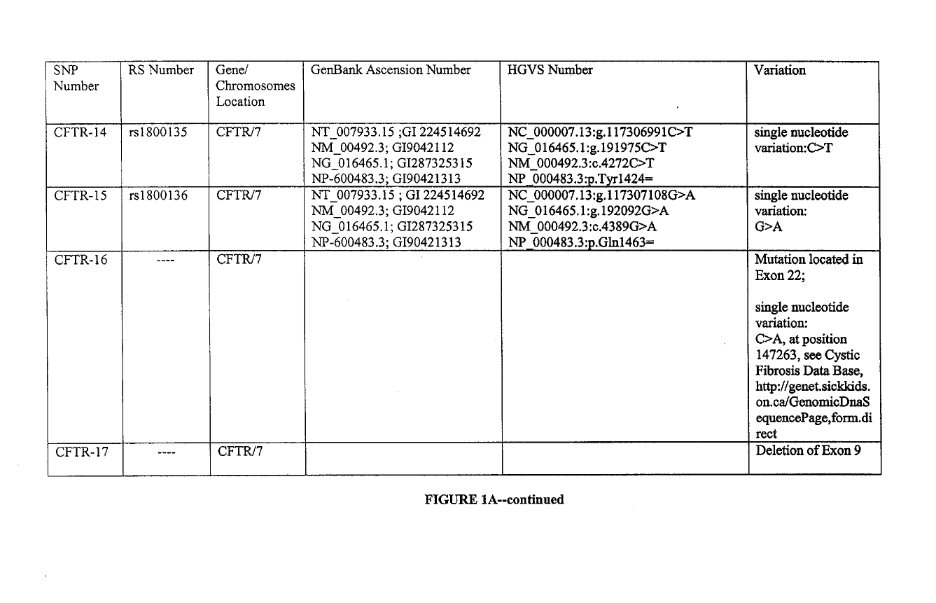

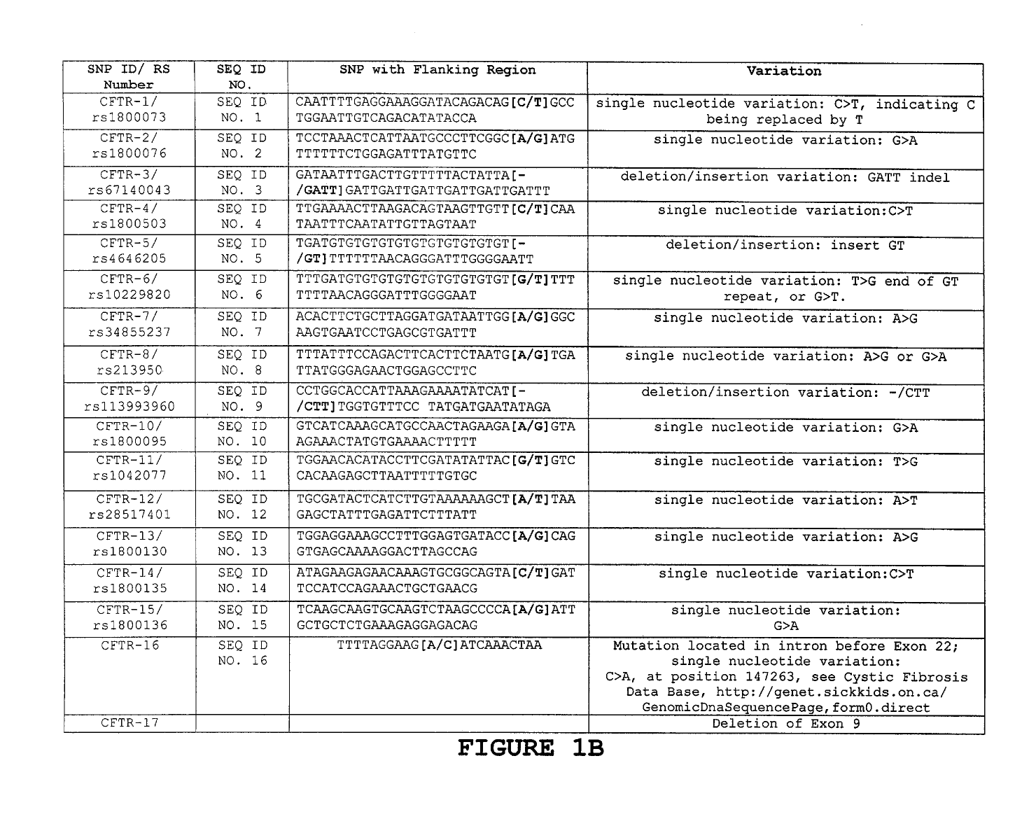

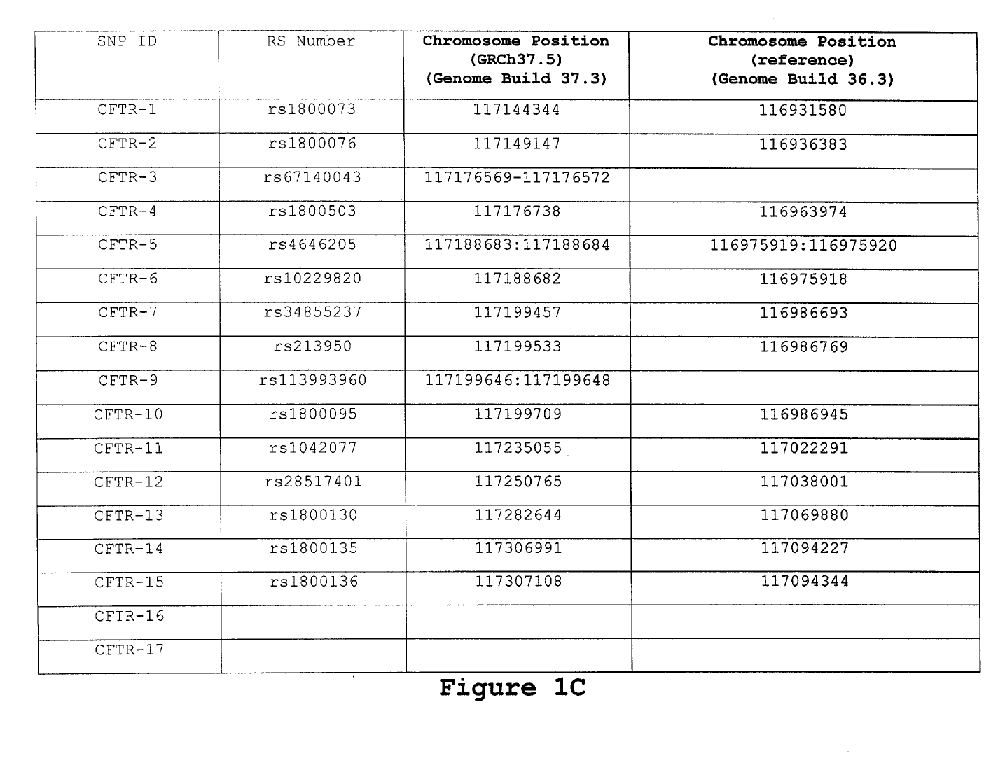

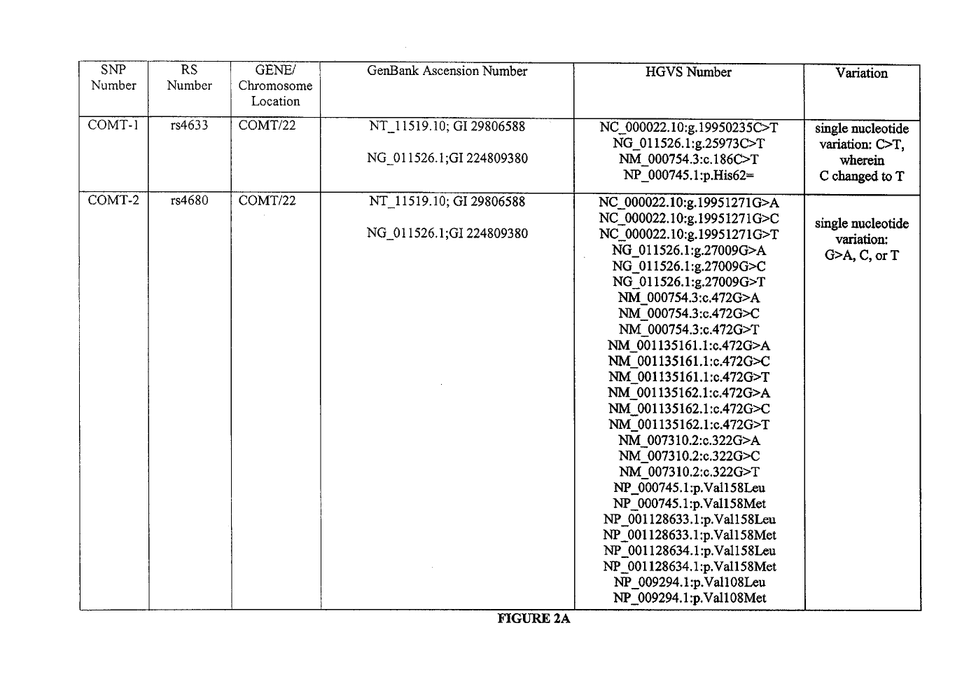

The inventors of the present invention discovered that mutations in the Cystic Fibrosis Transmembrane Conductance Regulator (CFTR) gene can be used to assess the risks associated with a person having or developing autism. The mutations include one or more SNPs located in the CFTR gene, including CFTR-1 (29328C>T), CFTR-2 (34131G>A), CFTR-3(61553_{—}61556delGATT), CFTR-4(61722C>T), CFTR-5(73667_{—}73668insGT), CFTR-6(73666T>G), CFTR-7(117199457A>G), CFTR-8(84517G>A), CFTR-9 (84630_{—}84632delCTT), CFTR-10(84693G>A), CFTR-11(120039T>G), CFTR-12(135749A>T), CFTR-13(167628A>G), CFTR-14 (191975C>T), CFTR-15(192092G>A), CFTR-16 (147263 C>A), CFTR-17 (Exon 19 deletion), see FIGS. 1A, 1B and 1C. FIG. 1A providing information relating to the identification of each of the SNPs, including HGVS number which provides the type of variation change associated with the gene and location in the gene in at the DNA level as well as at the mRNA and protein level. FIG. 1B provides the allele variation with flanking alleles. FIG. 1C describes chromosome position using Genome Build version 37.3 or 36.3. Except where noted, all information was obtained by the NCBI dbSNP database.

The inventors of the present invention discovered that mutations in the Cystic Fibrosis Transmembrane Conductance Regulator (CFTR) gene can be used to assess the risks associated with a person having or developing autism. Preferably, a predetermined number, defined as a threshold level, of mutations within the CFTR gene is detected within individuals. Determine a SNP variation within a certain number and not necessarily the specific SNP detected provides indication of risk association. Preferably, an individual's sample is tested to determine one or both alleles present/absent specific points within the CFTR gene A determination of the mutations including SNPs associated with CFTR-1 (29328C>T), CFTR-2 (34131G>A), CFTR-3(61553_{—}61556delGATT), CFTR-4(61722C>T), CFTR-5(73667_{—}73668insGT), CFTR-6(73666T>G), CFTR-7(117199457A>G), CFTR-8(84517G>A), CFTR-9 (84630_{—}84632delCTT), CFTR-10(84693G>A), CFTR-11(120039T>G), CFTR-12(135749A>T), CFTR-13(167628A>G), CFTR-14 (191975C>T), CFTR-15(192092G>A), CFTR-16 (147263 C>A), CFTR-17 (Exon 19 deletion), see FIG. 1.

While numerous factors may be critical in the development of autism, review of bacterial imbalances in the gastrointestinal track of children with autism indicated that an unusually high number were found to be positive for the bacteria Helicobacter pylori (H. pylori). Populations tested by the present inventor indicate that over 30% of the children were positive for H. pylori and up to 85% showed additional indicators suggesting H. pylori. While such a rate is typically seen in the adult population, the percentage of children testing positive is high. Moreover, the value may be higher as positive testing can be masked by the fact that H. pylori can hides in the mucosa. Epidemiological studies have shown that approximately one third of the adult population is infected with H. pylori in the United States, with a higher prevalence in developing countries. The rate of H. pylori is reported to increase at a rate of 1% a year, such that children under 12 have a rate of 5 to 10% in developed countries such as the US. Helicobacter pylori infection in children and this rate slowly increases over time to the reported rate of 30% in the adult population, see Shaman Rajindrajith, Niranga Manjuri Devanarayana, H Janaka de Silva, Helicobacter pylori infection in children, Sri Lanka Journal of Child health, 2009; 38(3): 86-88; Graham et al. Epidemiology of Helicobacter pylori in an asymptomatic population in the United States. Effect of age, race, and socioeconomic status, Gastroenterology. 1991 June; 100(6):1495-1501; Przybyszewska K. et al. Frequency of Helicobacter pylori infection in children under 4 years of age. Physiol Pharmacol. 2006 September; 57 Suppl 3:113-122. A study undertaken in Brazil indicates that this rate can be as high as 50%.

H. pylori is known to hide in the mucosa of infected cells of the GI tract, see S. Schreiber, The spatial orientation of Helicobacter pylori in the gastric mucus, PNAS, Apr. 6, 2004 vol. 101no. 14 5024-5029. One possible link between CFTR genes and autism may be a result from H. pylori having a greater ability to colonize the mucous layer of the GI in autistic children as compared to the non autistic population because such population has abnormal GI tract. In those suffering from cystic fibrosis, thick sticky mucous is produced in the lungs, creating problems in the lungs leading to lung infection. In an analogous fashion, it is believed that individuals with autism could have thicker mucous in the GI tract rather than thicker mucous in the lungs as seen in classic CF patients.

FIG. 1 provides identification for each CFTR related SNP. The Figure provides identification means through SNP identification, gene name and chromosome number location, RefSNP accession ID (rs numbers), identification from the public database GenBank (GenBank Ascension Number), and identification of the SNP using the Human Genome Variation Society identification (HGVS) of the SNPs. The HGVS naming system allows for identification of the SNP relative to their transcript sequences, genomic sequences, and/or their encoded protein sequences.

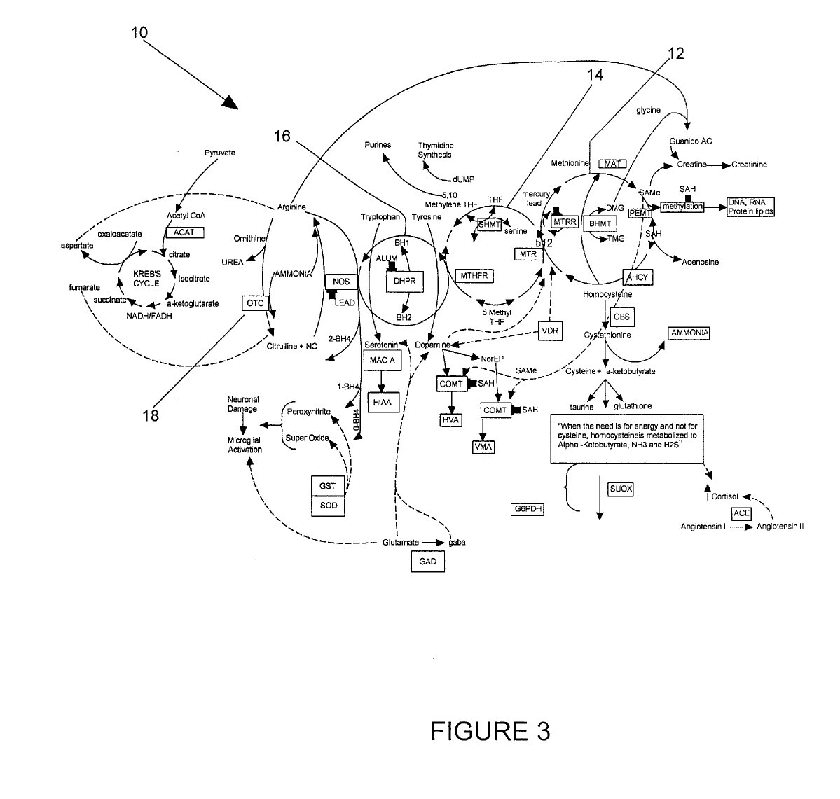

In addition to the CFTR based nucleotide variations, the present inventor determined that additional nucleotide variations in other genes play a role in autism. Specifically, several genes involved with the methylation pathway were determined to play a role in the assessment of individuals and their risk factors to developing autism. The methylation pathway describes the biochemical pathway that contributes to methylation, or addition of methyl groups to molecules. A properly functioning methylation pathway plays a role in different bodily functions, including but not limited to detoxification, immune function, maintenance of DNA, and energy production. For example, addition of methyl groups to DNA plays a vital role in the epigenetic regulation, which in turn determines whether a gene is expressed. As used herein, the term “methylation pathway” refers to a combination of four interrelated biochemical cycles, the methionine cycle, the folate cycle, the BH4 (biopertin) cycle, and the urea cycle. FIG. 3 illustrates the biochemical pathway for the methylation cycle 10 and the individual methionine cycle 12, the folate cycle 14, the BH4 (biopertin) 16 cycle, and the urea cycle 18. When the methylation pathway functions properly, various byproducts are produced that are used in other pathways to perform other important biological functions. However, when the methylation pathway is not functioning properly, several bodily functions do not perform efficiently and byproducts of the improperly functioning pathways can result in problems such as inflammation, increased vulnerability to infection, or decreased nerve transmission, resulting in disease conditions such as cardiovascular disease, cancer, diabetes, adult neurological disease, autism and other related diseases, and Alzheimer's disease.

Mutations within several genes associated with the methylation pathway can be used in combination with one or more CFTR mutations listed in FIG. 1 to assess the risks associated with a person having or developing autism. The genes include COMT (catechol-O-Methyltransferase, catalyzes the transfer of a methyl group from S-adenosylmethionine to catecholamines), VDR (Vitamin D Receptor, Taq and Fok binding sites) MAO A (Monoamine Oxidase A, enzyme active in serotonin breakdown), ACAT102 (Acetyl coenzyme A acetyltransferase, enzyme plays a role in cholesterol and other lipid balance), MTHFR (methylenetetrahydrofolate reductase, gene product playing a role in the methylation pathway by pulling homocysteine into the cycle) MTR (methionine synthase, regenerate and utilize B12 in the pathway and convert homocysteine to methionine), MTRR (methionine synthase reductase, regenerate and utilize B12 in the pathway and convert homocysteine to methionine) BHMT (betaine homocysteine methyltransferase, protein product playing a role in converting homocysteine to methionine), AHCY (S adenosylhomocysteine hydrolase, an enzyme responsible for converting s-adenosyl homocysteine (SAH) to adenosine and homocysteine, CBS (cystathionine β-synthase, key enzyme in homocysteine metabolism) SOUX (sulfite oxidase, terminal enzyme in the oxidative degradation pathway of sulfur-containing amino acids), SHMT (serine hydroxymethyltransferase, gene product helps in shifting the methylation cycle toward the building blocks for new DNA synthesis), NOS (nitric oxide synthase, enzyme which plays role in ammonia detoxification as part of the urea cycle) and PEMT (Phosphati-dylethanolamine-N-methyltransferase.

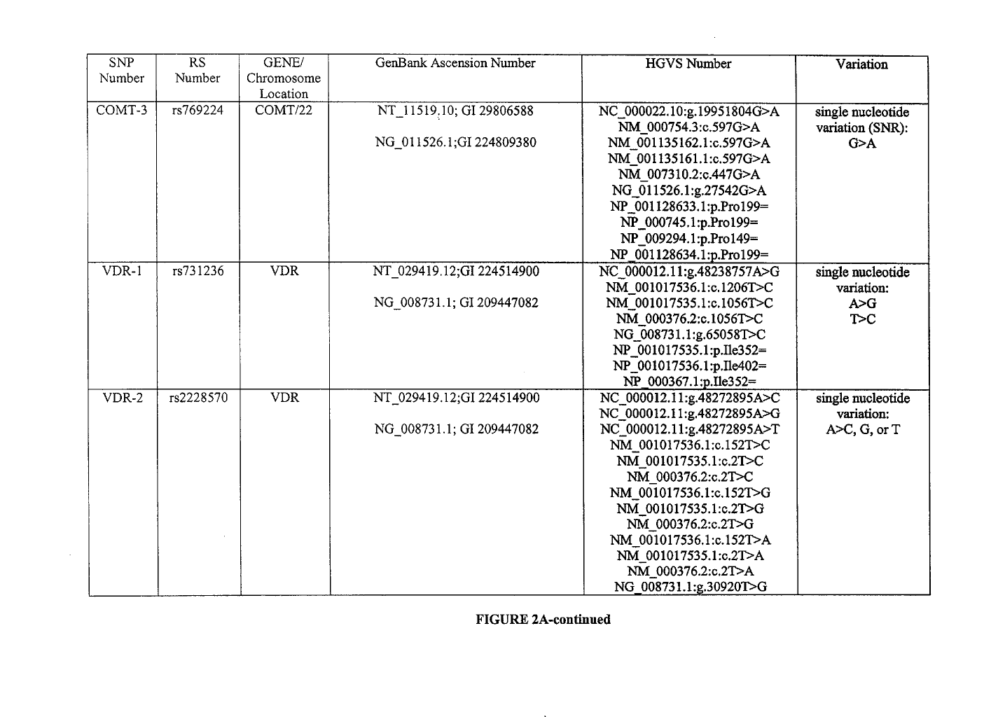

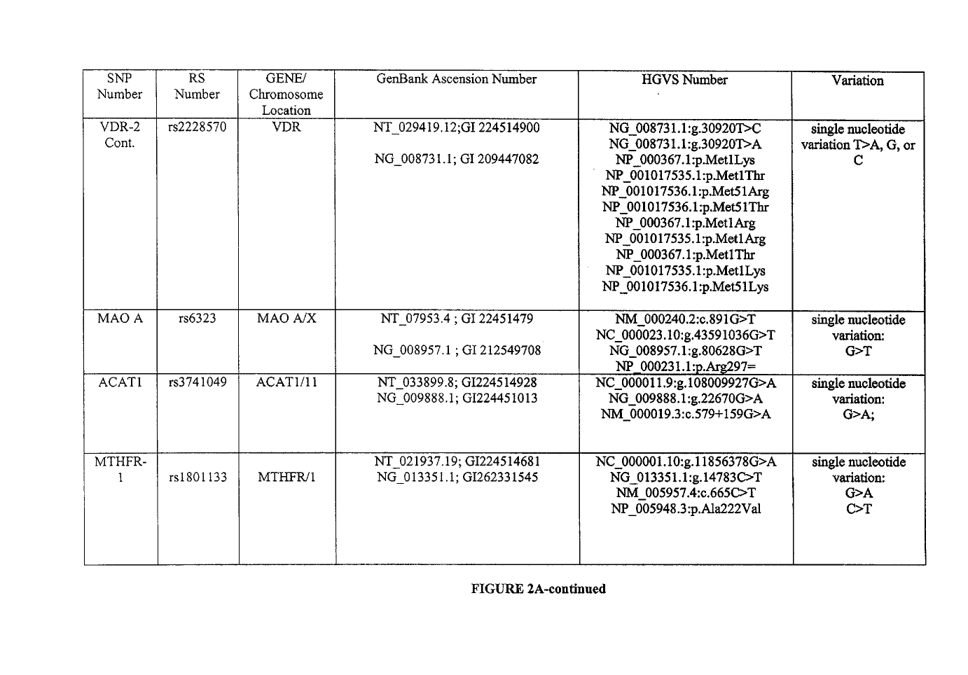

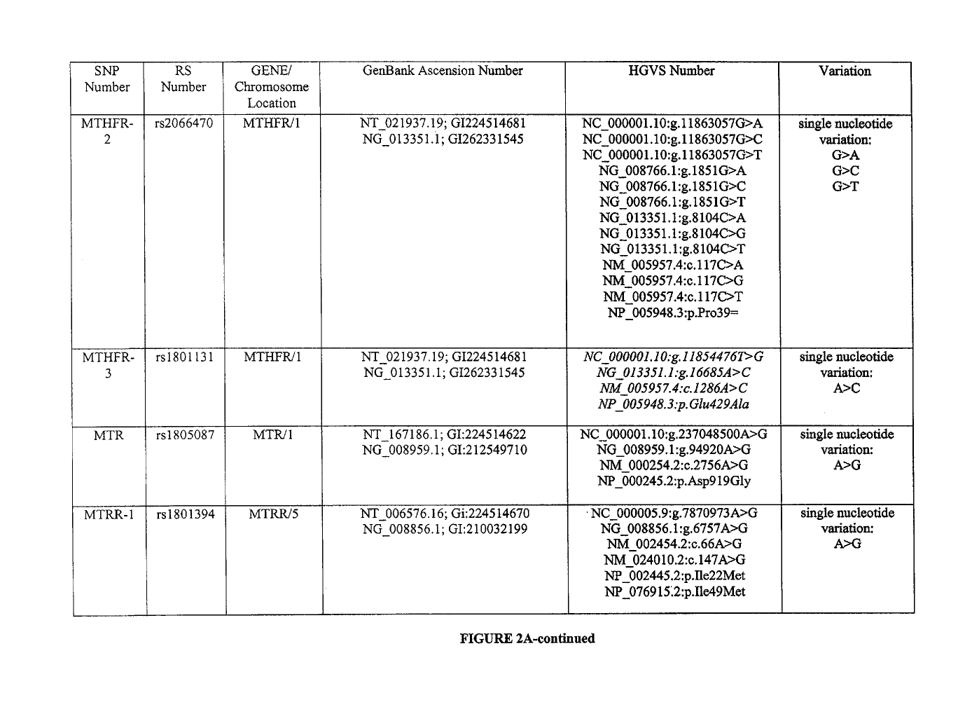

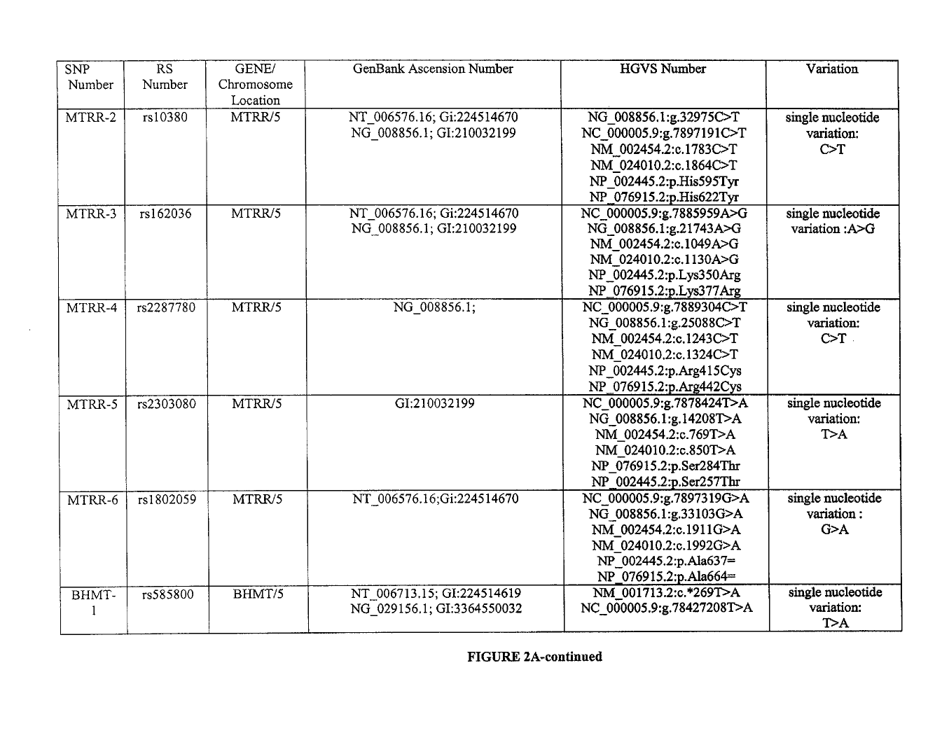

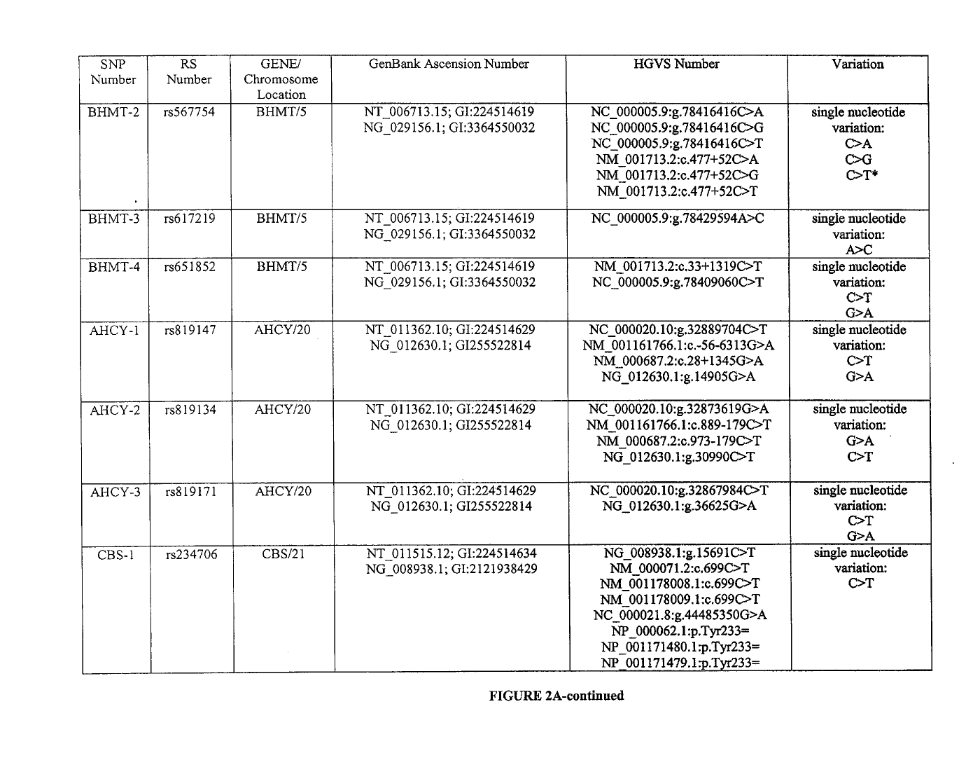

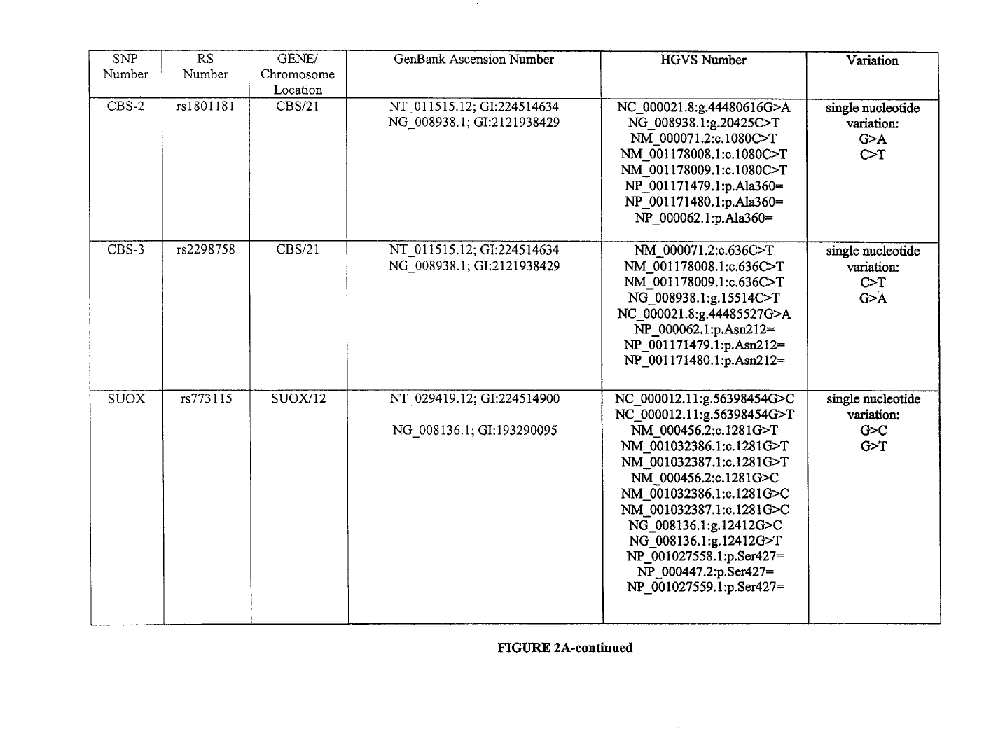

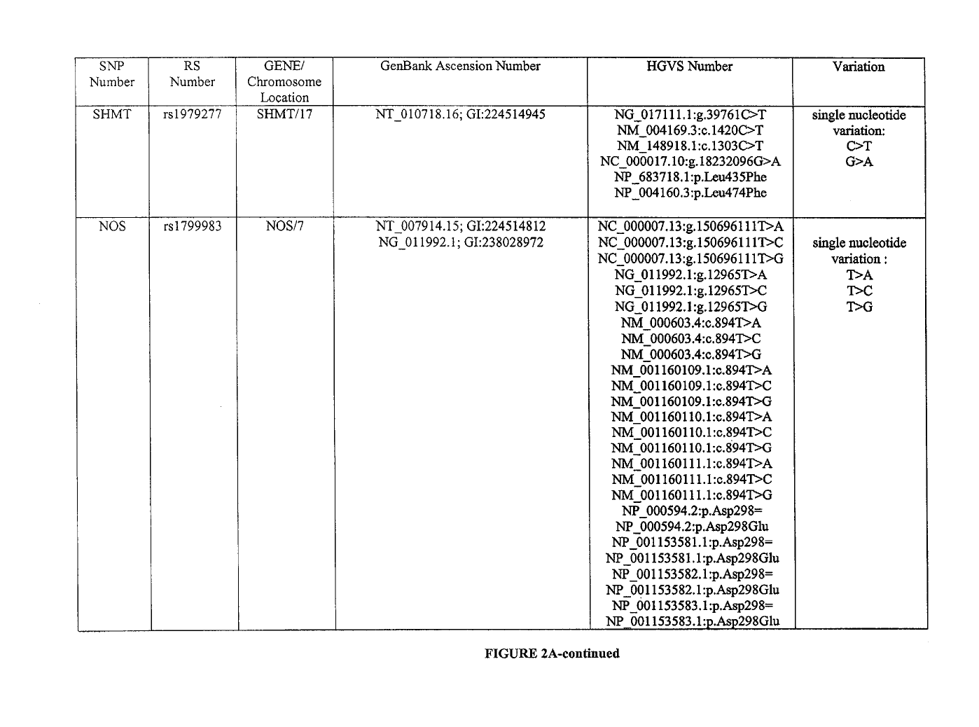

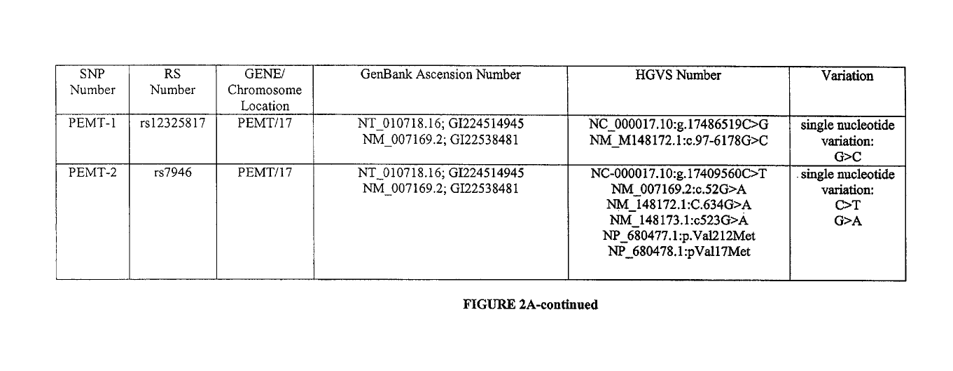

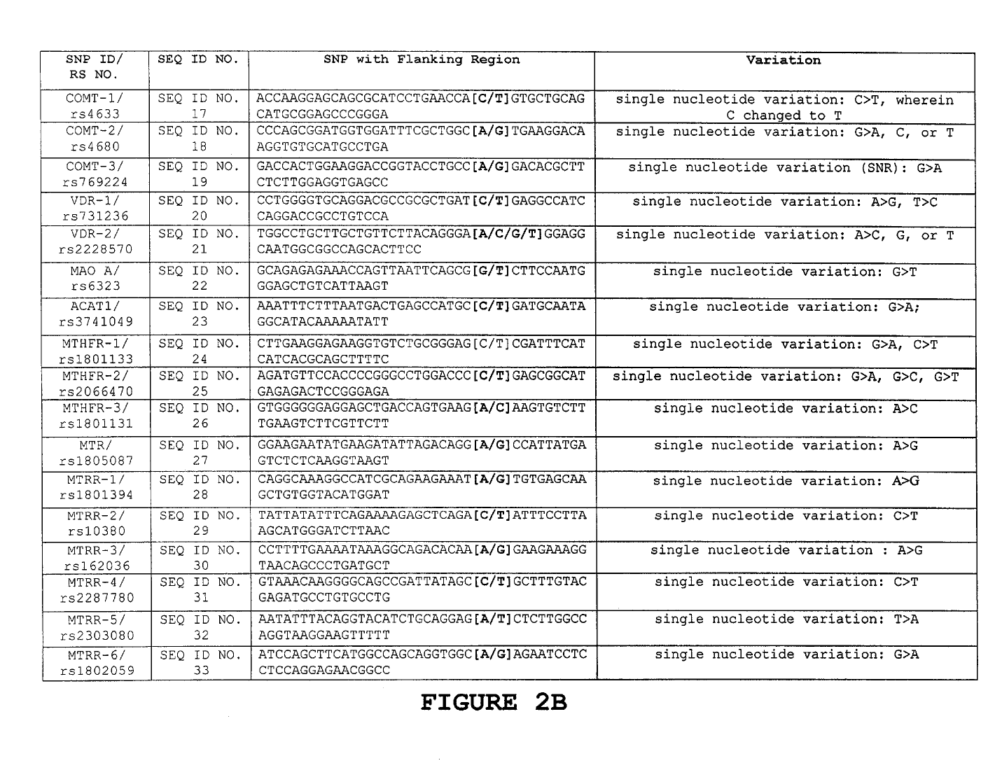

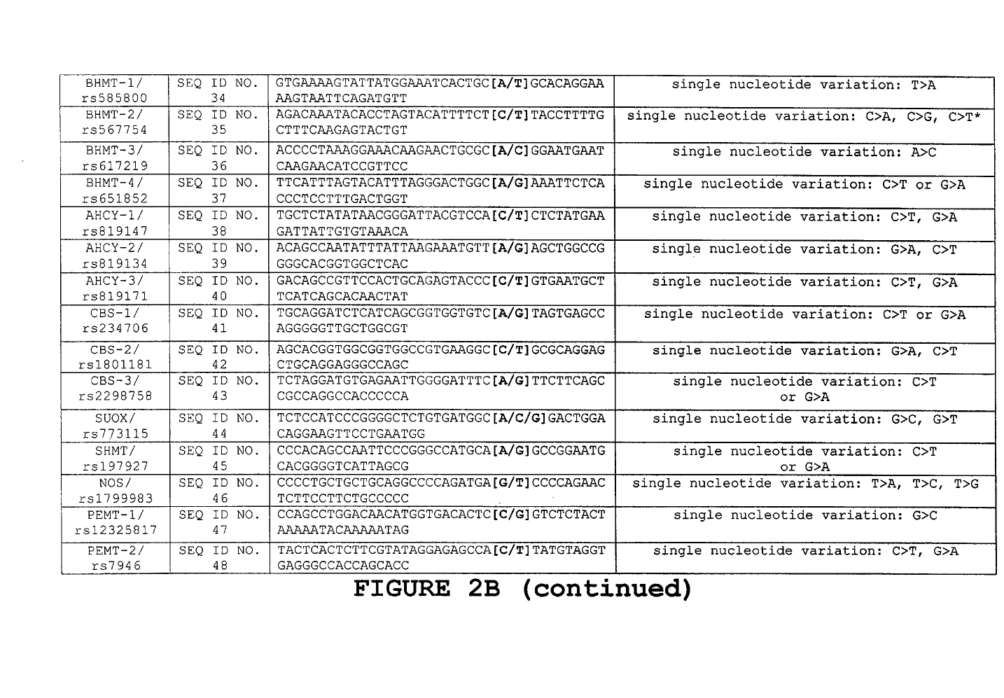

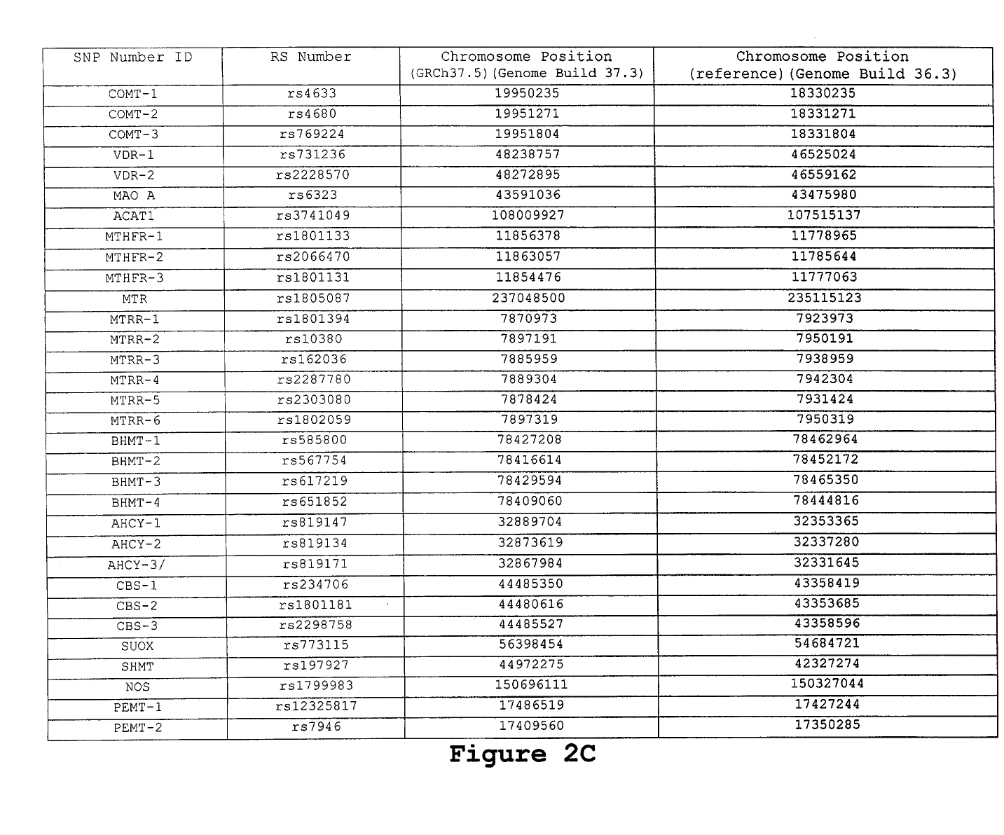

The specific nucleotide variations in the following one or more methylation pathway genes include, COMT-1, COMT-2, COMT61-3, VDR-1, VDR-2, MAO A, ACAT102-1, ACAT102-2, MTHFR-1, MTHFR-2, MTHFR-3, MTR, MTRR-1, MTRR-2, MTRR-3, MTRR-4, MTRR-5, MTRR-6, BHMT-1, BHMT-2, BHMT-3, BHMT-4, AHCY-1, AHCY-2, AHCY-3, CBS-i, CBS-2, CBS-3, SOUX, SHMT, NOS, PEMT-1, and PEMT-2, see FIG. 2 for SNP identification associated with these genes. FIG. 2A provides information relating to the identification of each of the SNPs associated with the methylation pathway, including HGVS number which provides the type of variation change associated with the SNP, and location in the gene at the DNA level, as well as at the mRNA and protein level. FIG. 2B provides the allele variation with flanking alleles. FIG. 2C describes chromosome position using Genome Build version 37.3 or 36.3.

The unique panel of CFTR SNPs, in combination with the collection of SNPs for the methylation cycle, is used to define a susceptibility to autism. Susceptibility to autism can be measured directly in terms of actual genetic polymorphisms in the CFTR SNPs as well as indirectly in terms of decreased methylation cycle function causing epigenetic functional issues in the CFTR gene.

Preferably, the testing method is performed on human subjects, but such testing methods could be performed on other mammals. Biological samples from human subjects may include blood, urine, semen, hair, mucosal cells/mucosal scraping, tissue biopsy or other tissues containing the nucleic acid to be analyzed. Nucleic acid utilized in accordance with the present invention can be DNA, genomic DNA, cDNA, RNA, hnRNA, and/or mRNA. Additionally, the proteins encoded by the nucleic acid having the SNPs may be used for analysis as well.

Preferably, the presence of one or more polymorphisms/mutations can be determined by extracting DNA from any tissue of the body. For example, blood can be drawn and DNA extracted from blood cells and analyzed. Since it is believed there are certain environmental occurrences that may lead to the development of autism, a test that determines the risk factors as early as possible would be useful. Accordingly, the present invention contemplates the use of the SNP panel as a prenatal risk assessment/diagnosis method by testing fetal cells, placental cells or amniotic cells for mutations in the gene.

Several methods known to one of skill in the art provide for the detection of specific alleles, and may include DNA sequencing, mini-sequencing, hybridization, restriction fragment analysis, or oligonucleotide ligation assay. For SNP genotyping technologies, a method for determining the presence of the type of base present on an allele and the method of reporting the allele signal through signal detection methods are utilized. General allele discrimination techniques include hybridization/annealing, primer extension, and enzyme cleavage, with each technique utilizing solution based assays or liquid/solid assays, such as microarray, see, Richard M. Twyman, Single Nucleotide Polymorphism (SNP) Genotyping Techniques—An Overview, in Encyclopedia of Diagnostic Genomics and Proteomics, ed. J. Fuchs and M. Podda, Marcel Dekker, Inc. N.Y. (2005).

Specific approaches for detecting sequence variations known to those skilled in the art can be used. Many such techniques are listed and briefly described in U.S. Patent Application 2011/0046094 and U.S. Patent Application 2010/0144776. Detection of the allele variants may require amplification techniques, such as polymerase chain reaction (PCR) or ligase chain reaction. Applicable diagnostic techniques include, but are not limited to, DNA sequencing including mini-sequencing, primer extension assays including allele-specific nucleotide incorporation assays and allele-specific primer extension assays, allele-specific oligonucleotide hybridization assays, electrophoretic comparison assays, such as pulsed field gel electrophoresis (PFGE) analysis, Southern blot analysis, single stranded conformation analysis (SSCA), denaturing gradient gel electrophoresis (DGGE), temperature gradient gel electrophoresis (TGGE), denaturing HPLC (DHPLC), analysis of cleavage of heteroduplex DNA, hybridization with allele-specific oligonucleotides (ASO), oligonucleotide ligation assays (OLA), PCR using allele-specific primers (ARMS), dot blot analysis, flap probe cleavage approaches, restriction fragment length polymorphism (RFLP), kinetic PCR, and PCR-SSCP, fluorescent in situ hybridization (FISH), and RNAse protection assays.

Array platforms, i.e. microchips used for genotyping purposes may also be applicable to the present invention. An array typically consists of thousands of distinct nucleotide probes which are built up in an array on a silicon chip. Nucleic acid to be analyzed is fluorescently labeled, and hybridized to the probes on the chip. Use of array platforms provides parallel processing of thousands of probes at once and can accelerate the analysis.

In an illustrative example, the method of determining whether an individual has an altered risk for developing autism in accordance with the present invention may include carrying out a process selected from primer extension assay, an allele-specific primer extensions assays, an allele specific nucleotide incorporation assay, an allele-specific oligonucleotide hybridization assay, a 5′ nuclease assay, an assay employing molecular beacons, and an oligonucleotide ligation assay.

Method of Use and CFTR/Methylation Panel Development: The novel panel and method of use in accordance with the present invention is modeled after genetic testing for other diseases. Specifically, the present inventor has linked the CFTR gene as a predictor for identifying those individuals with an increased or decreased risk of developing autism. The CFTR gene is located on chromosome 7 and contains 27 coding exons, with a genomic sequence of around 230 kB. The CFTR gene was positionally cloned in 1989, see Kerem, B. et al., Identification of the Cystic Fibrosis Gene: Genetic Analysis. Science 1989; 245:1073-1080; Riordin, J. R., et. al. Identification of the Cystic Fibrosis Gene: Cloning and Characterization of Complementary DNA. Science 1989; 245:1066-1073 (erratum 1989; 245:1437); Rommens, J. M. et. al. Identification of the Cystic Fibrosis Gene: Chromosome Walking and Jumping, Science 1989; 245:1059-1065.

CFTR gene mutation testing is a common testing method for cystic fibrosis, an autosomal recessive disorder affecting multiple organ systems. The leading cause of death for individuals suffering from cystic fibrosis is a progressive decline in pulmonary function resulting from airway damage caused by thickened secretions and chronic microbial infections. CF is characterized by substantial allelic heterogeneity. While there are a reported over 1,000 genetic mutations in the CFTR gene, the American College of Medical Genetics (ACMG) recommends testing for 23 common CFTR mutations, see American College of Medical Testing Genetics, Technical Standard and Guidelines for CFTR Mutation Testing, 2006 Edition, http://www.acmg.net/Pages/ACMG_Activities/stds-2002/cf.htm. The 23 mutations include the following: deltaF508, N1303K, A455E, deltaI507, R553X, R560T, 711+1G>T, G542X, 621+1G>T, R1162X, 1898+1G>A, G551D, R117H, G85E, 2184del, W1282X, 1717-1G>A, R2789+5G>A, 334W, R347P, 3849+10kbC>T, 3659delC, and 3120+1G>A.

No known association has been made between mutations in the CFTR gene and autism. In order to test such an association, an initial group of fifty individuals clinically diagnosed with autism were evaluated for the presence of the 23 common CFTR mutations listed above. Using these 23 SNPs, it was believed that approximately 89-95% of any CFTR mutations should have been detected if there was a close relationship between CFTR carrier status of one of the major CFTR mutations and autism. None of the 23 CFTR mutations were identified in the 50 individuals, autistic population tested, other than two control autistic samples with a known major CFTR mutation. In spite of this negative result and based on the premise that a relationship between CFTR mutations and autism exists, the entire CFTR gene was sequenced for 10 individuals with clinically diagnosed autism. Blood samples from those 10 individuals were tested at a CLIA approved laboratory (Seqwright, Houston, Tex.) for complete DNA sequencing for the CFTR gene.

Sequencing the individual patients revealed 17 minor CFTR mutations in this population. A relationship between the minor CFTR mutations that are not reported to affect the lungs, and the autistic population was determined. While not being bound by such a single theory, it s believed that the minor CFTR mutations create increased mucous in the GI tract rather than in the lungs. Thus the more standard CFTR SNPs are related to CF disease and thicker mucous in the lungs. Conversely the minor CFTR SNPs that have been found not to be an issue for CF disease are actually the issue in autism. This minor CFTR SNPs are proposed to cause thicker mucous in the GI tract which allows for the higher than expected frequency of H. pylori in the GI tract of autistic individuals.

What appears to be emerging is that susceptibility to autism may be related to the process involved with cystic fibrosis in the lungs. In classic cystic fibrosis, one or more of the more standard CFTR mutations is present. This allows for thicker mucous in the lungs, resulting in lung infection. In autism, however, the minor CFTR mutations were present (rather than the standard CFTR SNPs) and presumably thicker mucous in the GI tract (rather than in the lungs) such that it sets the stage for H. pylori infection in the GI tract at a much higher rate than predicted for children in the U.S. Additionally, the presence of H. pylori has been tied to the condition of sarcoidosis. While standard sarcoidosis affects the lungs, neurosarcoidosis is a related condition affecting the nervous system. Further studies are underway to investigate the possibility that H. pylori infection in autism (secondary to the presence of minor CFTR mutations) may allow for neurosarcoidosis which may also be a contributing factor in autism. Sarcoidosis and neurosarcoidosis are characterized by increased levels of eosinophils. Initial studies indicate that a large proportion of autistic children do show eosinophilia. The pathway to autism may also include an underlying susceptibility based on minor CFTR mutations, as well as methylation cycle mutations, as methylation cycle function controls CFTR activity. Susceptibility based on minor CFTR mutations and methylation cycle mutations could result in factors that contribute to development of autism, such as could allowing for thicker mucous in the GI tract which results in H. pylori infection, increased eosinophils, and neurosarcoidosis.

The genetic variation ranged from single nucleotide variations, i.e. allele variation of G changed to T or an insertion or deletion of one or more nucleotides. The minor nucleotide mutations were found within or near several Exons, see Table 3 for summary.

TABLE 1
Summary of CFTR gene SNP location

| | Mutation | |
|---|---|---|
| Exon | Details | Mutations Identified |
| 1 | No Mutations | |
| | Found | |
| 2 | Positive | rs1800073 |
| | Identification | Heterozygous mutation |
| | of Mutations | |
| 3 | Positive | rs1800076 |
| | Identification | Heterozygous mutation |
| | of Mutation | |
| 4 | No Mutations | |
| 5 | No Mutations | |
| 6 | No Mutations | |
| 7 | Positive | rs67140043 |
| | Identification | Mutation located in the intron before |
| | of Mutations | Exon 7, Heterozygous deletion of one |
| | | GATT repeat |
| | | rs1800503 |
| | | Mutation located in the intron after |
| | | Exon 7, Heterozygous mutation |
| 8 | No Mutations | |
| 9 | No Mutations | |
| 10 | Positive | rs4646205 |
| | Identification | Mutation located in the intron before |
| | of Mutations | Exon 10, Heterozygous insertion of one |
| | | TG repeat |
| | | rs10229820 |
| | | Mutation located in the intron before, |
| | | Exon 10, Heterozygous mutation |
| 11 | Positive | rs34855237 |
| | Identification | Mutation located in the intron before |
| | of Mutations | Exon 11, Heterozygous mutation |
| | | rs213950 |
| | | Homozygous mutation |
| | | rs113993960 |
| | | Heterozygous mutation deletion |
| | | rs1800095 |
| | | Heterozygous mutation |
| 12 | No Mutations | |
| 13 | No Mutations | |
| 14 | No Mutations | |
| 15 | Positive | rs1042077 |
| | Identification | Heterozygous/homozygous mutation |
| | of Mutations | |
| 16 | No Mutations | |
| 17 | No Mutations | |
| 18 | No Mutations | |
| 19 | Positive | rs28517401 |
| | Identification | This mutation is located in the intron |
| | of Mutations | after Exon 19, Heterozygous mutation |
| | Found | |
| 20 | No Mutations | |
| 21 | No Mutations | |
| 22 | Positive | Unknown |
| | Identification | This mutation is located in the intron |
| | of Mutations | before Exon 22,Heterozygous/homozygous |
| | Found | mutation |
| 23 | Positive | rs1800130 |
| | Identification | Heterozygous mutation |
| | of Mutations | |
| | Found tab | |
| 24 | No Mutations | |
| 25 | No Mutations | |
| 26 | No Mutations | |
| 27 | Positive | rs1800135 |
| | Identification | rs1800136 |
| | of Mutations | Heterozygous/homozygous mutation |
| | Found | |

Specific SNP analysis was initially run for 4 of these 16 mutations against an additional 30 blood samples taken from individuals diagnosed with autism. The sequencing methodology utilized the MassARRAY iPLEX™ platform for analyzing SNPs. Such platform combines the technologies of mass spectrometry and primer extension. PCR was performed initially to amplify the region surrounding the SNP. Primer extension was performed utilizing mass modified dideoxy nucleotides. All reactions were terminated after a single-base extension (SBE). A single post-PCR primer extension reaction generated diagnostic products that, based on their unique mass values, allowed discriminating between two alleles. A chip-based mass spectrometry was used for separation and analysis of the DNA analytes. The entire process was designed for complete automation including assay development, PCR setup, post-PCR treatment, nanoliter transfer of diagnostic products to silicon chips, serial reading of chip positions in the mass spectrometer, and final analytical interpretation.

The system uses mass spectrometry techniques, such as MALDI-TOF (matrix assisted laser desorption/ionization-time-of-flight mass spectrometry) to identify alleles on the basis of mass. Since allele calling depends on mass, it does not require expensive labeled primers and is also more reliable than other genotyping approaches. The average error rate reported in the laboratory with this method was <0.2% with high quality correctly quantitated DNA. The mass modified SBE products allow for use of highly stringent calling thresholds and performance at high plexing levels. The platform further utilized minimal DNA, 5-10 ng of DNA per set of multiplexed assays and provided call rates of greater than 95% on high quality samples that are accurately quantitated.

Quality control was achieved by typing internal positive control samples of known genotypes with no template controls and by QC replicate samples (5%-10% of total). Genotyping plates were reviewed for results from positive- and negative-DNA control wells organized in specific patterns to assist in the QC process and to ensure correct plate orientations during processing and data review. In initial assay development, DNAs from 20 individuals from Coriell's Polymorphism Discovery Resource were used. SNP assays with genotype call rates of <75% are excluded or redesigned. SNP assays are also checked for Hardy-Weinberg equilibrium (HWE), and SNPs out of HWE (P<0.01) are excluded or re-genotyped.

The SNPs important for the diagnosis and/or determination of at risk individuals identified in FIG. 1 were based on the initial screening from the 10 individuals tested. The initial results are shown in Tables 2-12 for the Patients #1-10. All of the autistic individuals had one or more of these minor CFTR mutations. In some cases, additional patient samples were tested for the presence or absence of several SNPs. Sample 10 was a clinically diagnosed autistic individual with a known major CFTR mutation.

TABLE 2
Testing Results for SNP Mutations in CFTR-1.

| | CFTR-1 |
|---|---|
| Patient | Variation: C |
| Identification | replaced by T |
| Sample I.D #1 | C/C |
| Sample I.D #2 | C/C |
| Sample I.D #3 | C/C |
| Sample I.D #4 | C/C |
| Sample I.D #5 | C/C |
| Sample I.D #6 | C/C |
| Sample I.D #7 | C/C |
| Sample I.D #8 | C/T |
| Sample I.D #9 | C/T |
| Sample I.D #10 | C/C |

Referring to Table 2, the results for CFTR-1 is shown, with Sample I.D. numbers 8 and 9 having the single nucleotide variation, C being replaced by a T. Note that the single nucleotide variation resulted in a heterozygous mutation.

TABLE 3
Testing Result for SNP Mutations in CFTR-2.

| | CFTR-2 |
|---|---|
| Patient | Variation: G |
| Identification | replaced by A |
| Sample I.D #1 | G/A |
| Sample I.D #2 | G/G |
| Sample I.D #3 | G/G |
| Sample I.D #4 | G/G |
| Sample I.D #5 | G/G |
| Sample I.D #6 | G/G |
| Sample I.D #7 | G/G |
| Sample I.D #8 | G/G |
| Sample I.D #9 | G/G |
| Sample I.D #10 | G/G |

Table 3 shows the results for CFTR-2. As shown in the Table, Sample I.D. 1 has the single nucleotide variation, G being replaced by A. Note that the single nucleotide variation resulted in a heterozygous mutation.

TABLE 4
Testing Result for SNP Mutations in CFTR-3 and CFTR-4.

| | CFTR-3 | CFTR-4 |
|---|---|---|
| Patient | Variation: GATT | Variation: C |
| Identification | delete | replaced by T |
| Sample I.D #1 | (GATT)₇/(GATT)₇ | C/C |
| Sample I.D #2 | (GATT)₇/(GATT)₆ | C/T |
| Sample I.D #3 | (GATT)₇/(GATT)₇ | C/C |
| Sample I.D #4 | (GATT)₇/(GATT)₆ | C/T |
| Sample I.D #5 | (GATT)₇/(GATT)₇ | C/C |
| Sample I.D #6 | (GATT)₇/(GATT)₆ | C/C |
| Sample I.D #7 | (GATT)₇/(GATT)₇ | C/C |
| Sample I.D #8 | (GATT)₇/(GATT)₆ | C/T |
| Sample I.D #9 | (GATT)₇/(GATT)₆ | C/T |
| Sample I.D #10 | (GATT)₇/(GATT)₆ | C/T |

Table 4 shows the results for CFTR-3 (left column) and FTR-4 (right column). As shown in the Table for CFTR-3, Sample I.D. 2, 4, 6, and 8-10 each have a heterozygous deletion of one of the GATT repeats. Of note, the GATT deletion is suppose to be an in absolute linkage disequilibrium with the delta 508 SNP. Most of the patients had the GATT deletion but not the delta 508 mutation. In our case, the GATT deletion is not linked to delta 508, making identification of this SNP in this population significant and unique.

With respect to CFTR-4, Sample I.D. 2, 4, and 8-10 each have a heterozygous variation, C being replaced by T.

TABLE 5
Testing Result for SNP Mutations in CFTR-5 and
CFTR-6, and CFTR-17.

| | | CFTR-6 |
|---|---|---|
| | CFTR-5 | Variation: T |
| Patient | Variation: TG | replaced by G or |
| Identification | insert | G replaced by T |
| Sample I.D #1 | (TG)₁₁/TG₁₁ | G/G |
| Sample I.D #2 | (TG)₁₁/TG₁₁ | G/T |
| Sample I.D #3 | (TG)₁₁/TG₁₁ | G/G |
| Sample I.D #4 | indel | |
| Sample I.D #5 | (TG)₁₀/TG₁₀ | G/G |
| Sample I.D #6 | indel | |
| Sample I.D #7 | (TG)₁₁/TG₁₁ | G/G |
| Sample I.D #8 | (TG)₁₁/TG₁₁ | G/G |
| Sample I.D #9 | (TG)₁₁/TG₁₁ | G/G |
| Sample I.D #10 | (TG)₁₁/TG₁₁ | G/T |

Table 5 shows the results for CFTR-5 (left column) and CFTR-6 (right column). As shown in the Table for CFTR-5, Sample I.D. 1, 2, 3, 7, 8, 9, and 10 had homozygous mutation deletion of one of the TG repeats. With respect to CFTR-6, Sample I.D. 2 and 10 each have a heterozygous variation, T being replaced by G and Sample I.D. 1, 3, 5, 7, 8, and 9 each have a homozygous variation.

The combination of the length of the TG span (CFTR 5) and the number of T's after the TG span (CFTR 6) define whether or not Exon 9 will be completely skipped. Skipping of Exon 9 seriously impacts the function of the CFTR gene. The longer the TG stretch and the shorter the T region the greater the issue. For example, (TG)12 has a 6 fold greater likelihood of lacking Exon 9. (TG)11 has a 2.8 fold greater likelihood of lacking Exon 9 as compared to (TG)10. The number of TTs following the TG repeat region plays a role in CFTR activity such that the combination of TG number and T number defines whether or not Exon 9 will be skipped. Skipping of Exon 9 seriously impairs CFTR function. Thus CFTR 6 which is a change from the last base in the TG sequence affects the total number of TG segments and Ts. When the base for CFTR 6 is a “G” instead of a “T” it lengthens the TG span and shortens the T span such that the TG span is longer and the T span is shorter thus increasing the likelihood that Exon 9 will be skipped. Accordingly, the degree of Exon skipping is inversely correlated with the length of the (T)n tract so that transcripts derived from genes that carry 5 thymidines (5T) at this locus have the highest levels of Exon 9 skipping, whereas those with 7 or 9 thymidines (7T or 9T, respectively) exhibit progressively lower levels of skipping (Chu et al, 1993).

Table 5 further illustrates that 20% of the samples, Sample I.D. 4 and 6 contained the deletion of Exon 9.

TABLE 6
Testing Result for SNP Mutations in CFTR-7 and CFTR-8.

| | | CFTR-8 |
|---|---|---|
| | CFTR-7 | Variation: G |
| Patient | Variation: A | replaced by A or |
| Identification | replaced by G | A replaced by G |
| Sample I.D #1 | A/A | G/G |
| Sample I.D #2 | A/G | G/A |
| Sample I.D #3 | A/A | G/G |
| Sample I.D #4 | no coverage | A/A |
| Sample I.D #5 | A/A | A/A |
| Sample I.D #6 | A/G | G/A |
| Sample I.D #7 | A/A | G/G |
| Sample I.D #8 | A/G | G/A |
| Sample I.D #9 | A/G | G/A |
| Sample I.D #10 | no coverage | G/A |

Table 6 shows the results for CFTR-7 (left column) and CFTR-8 (right column). As shown in the Table for CFTR-7, Sample I.D. 2, 6, 8 and 9 had a heterozygous mutation deletion, A being replaced by G. With respect to CFTR-8, Sample I.D. 2, 6 and 8, 9, and 10 each have a heterozygous variation, A being replaced by G and Sample I.D. 1, 3, and 7 each have a homozygous variation, A being replaced by G.

TABLE 7
Testing Result for SNP Mutations in CFTR-9 and CFTR-10.

| | CFTR-9 | CFTR-10 |
|---|---|---|
| Patient | Variation: deletion | Variation: G |
| Identification | of CTT | replaced by A |
| Sample I.D #1 | CTT/CTT | G/G |
| Sample I.D #2 | CTT/CTT | G/G |
| Sample I.D #3 | CTT/CTT | G/G |
| Sample I.D #4 | CTT/CTT | G/G |
| Sample I.D #5 | CTT/CTT | G/A |
| Sample I.D #6 | CTT/CTT | G/G |
| Sample I.D #7 | CTT/CTT | G/G |
| Sample I.D #8 | CTT/CTT | G/G |
| Sample I.D #9 | CTT/CTT | G/G |
| Sample I.D #10 | CTT/delCTT | G/G |

Table 7 shows the results for CFTR-9 (left column) and CFTR-10 (right column). As shown in the Table for CFTR-9, Sample I.D. 10 had a heterozygous mutation deletion of CTT. With respect to CFTR-10, Sample I.D. 5 had a heterozygous variation, G being replaced by A.

TABLE 8
Testing Results for SNP Detection in CFTR-11.

| | CFTR-11 |
|---|---|
| Patient | Variation: T |
| Identification | replaced by G |
| Sample I.D #1 | T/T |
| Sample I.D #2 | T/T |
| Sample I.D #3 | T/T |
| Sample I.D #4 | T/G |
| Sample I.D #5 | G/G |
| Sample I.D #6 | T/G |
| Sample I.D #7 | T/T |
| Sample I.D #8 | T/T |
| Sample I.D #9 | T/T |
| Sample I.D #10 | T/T |

Table 8 shows the results for CFTR-11. As shown in the Table for CFTR-11, Sample I.D. 4 and 6 had a heterozygous mutation, T being replaced by G and Sample I.D. 5 had a homozygous variation, T being replaced by G.

TABLE 9
Testing Results for SNP Detection in CFTR-12.

| | CFTR-12 |
|---|---|
| Patient | Variation: A |
| Identification | replaced by T |
| Sample I.D #1 | A/A |
| Sample I.D #2 | A/A |
| Sample I.D #3 | — |
| Sample I.D #4 | A/A |
| Sample I.D #5 | A/A |
| Sample I.D #6 | A/T |
| Sample I.D #7 | A/A |
| Sample I.D #8 | A/A |
| Sample I.D #9 | A/A |
| Sample I.D #10 | A/A |

Table 9 shows the results for CFTR-12. As shown in the Table, Sample I.D. 6 had a heterozygous mutation, A being replaced by T.

TABLE 10
Testing Results for SNP Detection in CFTR-13

| | CFTR-13 |
|---|---|
| Patient | Variation: A |
| Identification | replaced by G |
| Sample I.D #1 | A/A |
| Sample I.D #2 | A/A |
| Sample I.D #3 | A/G |
| Sample I.D #4 | A/A |
| Sample I.D #5 | A/A |
| Sample I.D #6 | A/A |
| Sample I.D #7 | A/A |
| Sample I.D #8 | A/A |
| Sample I.D #9 | A/A |
| Sample I.D #10 | A/A |

Table 10 shows the results for CFTR-13. As shown in the Table, Sample I.D. 3 had a heterozygous mutation, A being replaced by G.

TABLE 11
Results of Detection of SNP in CFTR-14 and CFTR-15.

| | CFTR-14 | CFTR-15 |
|---|---|---|
| Patient | Variation: C | Variation: G |
| Identification | replaced by T | replaced by A |
| Sample I.D #1 | C/C | G/G |
| Sample I.D #2 | C/C | G/G |
| Sample I.D #3 | C/C | G/A |
| Sample I.D #4 | C/C | G/A |
| Sample I.D #5 | C/C | A/A |
| Sample I.D #6 | C/C | A/A |
| Sample I.D #7 | C/C | G/G |
| Sample I.D #8 | C/C | G/G |
| Sample I.D #9 | C/C | G/G |
| Sample I.D #10 | C/C | G/G |

Table 11 shows the results for CFTR-14 (left column) and CFTR-15 (right column). While the initial sample test population, i.e. the 10 sample sequence group, indicated none of the samples being positive for CFTR-14, testing on additional patients indicated positive identification in 10% of the sample population. As shown in the Table for CFTR-15, Sample I.D. 3 and 4 had a heterozygous mutation, G being replaced by A, and Sample I.D. 5 and 6 had a homozygous variation, G being replaced by A.

TABLE 12
Results of Detection of SNP in CFTR-16.

| | CFTR-16 |
|---|---|
| Patient | Variation: C |
| Identification | replaced by A |
| Sample I.D #1 | C/C |
| Sample I.D #2 | C/C |
| Sample I.D #3 | C/C |
| Sample I.D #4 | C/A |
| Sample I.D #5 | A/A |
| Sample I.D #6 | C/C |
| Sample I.D #7 | C/C |
| Sample I.D #8 | C/C |
| Sample I.D #9 | C/C |
| Sample I.D #10 | C/C |

Table 12 shows the results for CFTR-16. As shown in the Table, Sample I.D. 4 had a heterozygous mutation, C being replaced by A, and Sample I.D. 5 had a homozygous mutation, C being replaced by A.

In addition to the 16 SNPs described above, the panel or method of method of determining whether an individual has an altered risk for developing autism may also include identification of SNP 17, the deletion of Exon 9 (SEQ ID NO.49 GATTTCTTACAAAAGCAAGAATATAAGACATTGGAATATAACTTAACGACTACAGA AGTAGTGATGGAGAATGTAACAGCCTTCTGGGAGGAG, positions 61,922 to 62,014, see demonic DNA sequence from Cystic Fibrosis Mutation Database, www.genet.sickkkids.on.ca/GenomicDnaSequencePage.form0.direct), the contents of which are incorporated herein by reference). Detection of Exon 9 skipping can be determined using the following process: Long-range PCR was performed across CFTR intron 9 using primer 9i5, see Zielenski et al., Genomic DNA sequence of the cystic fibrosis transmembrane conductance regulator (CFTR) gene. Genomics. 1991:10:214-228., located in intron 8 upstream from the (TG)ₘ(T)ₙ site, together with a reverse primer located at the end of exon (5′TGCTTTGATGACGCTTCTGTAT-3′) (SEQ ID NO: 49) and using 200 ng of genomic DNA from the patient. Nested PCR was performed to amplify the polypyrimidine sequence with previous exported primers RF9 and RR9, see Radpour et al, Two novel missense and pone novel nonsense CFTR mutations in Iranian males with congenital bilateral absence of the vas deferens. Mol Hum Reprod. 2006a; 12:717-721. The nested PCR conditions were as follows: denaturation at 95° C. for 30 seconds, annealing at 55° C. for 30 seconds, and extension at 74° C. for 40 seconds, for 32 cycles. Nested PCR products were digested with Xmn I and visualized on a 12% nondenaturing polyacrylamide gel.

These findings were significant for several reasons. First, no mutation was found when screening for the most common classical CFTR mutations that represent 89-95% of all CF mutations in the population. More importantly, despite not finding the 23 major mutations, the autistic population tested showed 100% of the samples having one or more of these minor mutations. These minor mutations are considered to be present at a level of less than 1%, with only a limited number of mutations occurring at frequencies >1% in any particular population, see Technical Standards for CFTR mutation Guidelines, American College of Medical Genetics, http://www.acmg.net/Pages/ACMG_Activities/stds-2002/cf.htm. Thus, 100% of the autistic population tested positive for CFTR mutations only found in less than 1% of the population and in addition this same autistic population tested negative for CFTR mutations representing up to 95% of those found in the general public. The pattern of CFTR SNPs in the autistic population is therefore unique and distinct from that of the general population.

While the present invention may include a set of SNPs as a method of determining genetic signature indicative for, or diagnostic of, autism containing 17 SNPs listed in FIG. 1 and the 30 SNPs listed in FIG. 2, the entire panel need not be used in each method or panel development. Accordingly, the method of determining whether a human has an altered risk for, or diagnosing of autism according to the present invention may include the use of 2 or more SNPs, 3 or more SNPs, 4 or more SNPs, 5 or more SNPs, 6 or more SNPs, 7 or more SNPs, 8 Or more SNPs, 9 or more SNPs, 10 or more SNPs, 11 or more SNPs, 12 or more SNPs, 13 or more SNPs, 12 or more SNPs, 13 or more SNPs, 14 or more SNPs, 15 or more SNPs, or 16 or more SNPs, or 17 or more SNPs selected from CFTR-1 (29328C>T), CFTR-2 (34131G>A), CFTR-3 (61553_{—}61556delGATT), CFTR-4 (61722C>T), CFTR-5 (73667_{—}73668insGT), CFTR-6 (73666T>G), CFTR-7 (117199457A>G), CFTR-8 (84517G>A), CFTR-9 (84630_{—}84632delCTT), CFTR-10 (84693G>A), CFTR-11 (120039T>G), CFTR-12 (135749A>T), CFTR-13 (167628A>G), CFTR-14 (191975C>T), CFTR-15 (192092G>A), CFTR-16 (147263 C>A), CFTR-17 (Exon 19 deletion).

The method of determining whether a human has an altered risk for developing autism may include identifying from a sample one or more SNPS, 2 or more SNPs, 3 or more SNPS, 4 or more SNPs, 5 or more SNPs, 6 or more SNPs, 7 or more SNPs, 8 or more SNPs, 9 or more SNPs, 10 or more SNPs, 11 or more SNPs, 12 or more SNPs, 13 or more SNPs, 12 or more SNPs, 13 or more SNPs, 14 or more SNPs, 15 or more SNPs, or 16 or more SNPs, 17 or more SNPs, 18 or more SNPS, 19 or more SNPs, 20 or more SNPs, 21 or more SNPs, 22 or more SNPs, 23 or more SNPs, 24 or more SNPs, 25 or more SNPs, 26 or more SNPs, 27 or more SNPs, 28 or more SNPs, or more SNPs, 30 or more SNPs selected from the group comprising of COMT-1, COMT-2, COMT1-3, VDR-1, VDR-2, MAO A, ACAT102-1, ACAT102-2, MTHFR-1, MTHFR-2, MTHFR-3, MTR, MTRR-1, MTRR-2, MTRR-3, MTRR-4, MTRR-5, MTRR-6, BHMT-1, BHMT-2, BHMT-3, BHMT-4, AHCY-1, AHCY-2, AHCY-3, CBS-1, CBS-2, CBS-3, SOUX, SHMT, NOS, PEMT-1, and PEMT-2 in combination with CFTR SNPs.

The exact number of SNPs within a panel and/or method therefore can be varied, with a variety of subset panels being created depending on the need of the individual being tested. The precise number utilized within the method in accordance with the present invention and/or in the panel can vary depending on a variety of factors and the information needed, including but not limited to the time of testing, i.e. initial screening versus follow-up testing, population tested, i.e. those who have been diagnosed with autism versus those suspected of having autism, or pre/post treatment regiments.

While a preferred example may include all the CFTR genes identified in FIG. 1, other combinations may be used. For example any 4 or 5 CFTR related SNPS may be sued in combination with any 4 or 5 methylation related SNPs. Table 15A illustrates a panel containing four CFTR gene SNPs, CFTR 2, 4, 7 and 8. Table 15B illustrates the results for a SNP Panel which includes the SNPs in Table 15A in combination with additional methylation gene SNPs including AHCY-1, AHCY-2, AHCY-3, BHMT-1, BHMT-2, BHMT-3, CBS-1, CBS-2, CBS-3, COMT-2, MTHFR-1, MTHFR-2, MTR, MTRR-1, MTRR-2, MTRR-3, MTRR-4, MTRR-5, MTRR-6, NOS, SHMT, SOUX, VDR-1 AND VDR-2.

TABLE 15A
4 CFTR SNP Panel

| | Number of | |
|---|---|---|
| SNP ID | Individuals Tested | Results |
| CFTR 2 | 39 | 2 positive for SNP |
| | | (all heterozygous) |
| CFTR 4 | 39 | 9 positive for SNP |
| | | (all heterozygous) |
| CFTR 7 | 37 | 15; 12 |
| | | heterozygous and 3 |
| | | homozygous |
| CFTR 8 | 39 | 30; 15 |
| | | heterozygous, 15 |
| | | homozygous |

TABLE 15B
4 CFTR SNP Panel in Combination with Methylation Genes SNPs.

| | | Results | |
|---|---|---|---|
| | | Total Number | |
| | | testing positive | |
| | | for SNP; (total | |
| | Number of | heterozygous/total | |
| SNP ID | Individuals Tested | homozygous) | |
| CFTR 2 | 39 | 2 positive for SNP | |
| | | (all heterozygous) | |
| CFTR 4 | 39 | 9 positive for SNP | |
| | | (all heterozygous) | |
| CFTR 7 | 37 | 15; | (12/3) |
| CFTR 8 | 39 | 30; | (15/15) |
| ACAT | 39 | 13; | (10/3) |
| AHCY-1 | 39 | 11; | (10/1) |
| AHCY-2 | 39 | 11; | (10/1) |
| AHCY-3 | 39 | 11; | (10/1) |
| BHMT-1 | 39 | 17; | (14/3) |
| BHMT-2 | 39 | 20; | (12/8) |
| BHMT-3 | 39 | 21; | (12/9) |
| BHMT-4 | 39 | 26; | (18/8) |
| CBS-1 | 39 | 18; | (15/3) |
| CBS-2 | 39 | 26; | (20/6) |
| CBS-3 | 15 | 2; | (2/0) |
| COMT-2 | 39 | 30; | (19/11) |
| MTHFR-1 | 39 | 24; | (20/4) |
| MTHFR-2 | 39 | 3; | (3/0) |
| MTR | 39 | 12; | (10/2) |
| MTRR-1 | 39 | 33; | (22/11) |
| MTRR-2 | 39 | 6; | (6/0) |
| MTRR-3 | 39 | 6; | (5/1) |
| MTRR-4 | 39 | 6; | (6/0) |
| MTRR-5 | 39 | 6; | (6/0) |
| MTRR-6 | 39 | 6; | (6/0) |
| NOS | 39 | 18; | (14/4) |
| SHMT | 24 | 14; | (11/3) |
| SOUX | 39 | 2; | (1/1) |
| VDR-1 | 39 | 36; | (20/16) |
| VDR-2 | 39 | 29; | (18/11) |

Additional sample testing indicated that a mutations or variations, whether homozygous or heterozygous, was found in approximately 26% of a test sample for COMT-1, 5% of a test sample for COMT-3, 75% of a test sample for MAO A, 53% of a test sample for MTHFR-3, 86% of a test sample for PEMT-1, and 82% of a test sample for PEMT-2. The composition of CFTR mutations and methylation mutations indicates that 100% of the individuals with autism have one or more CFTR and methylation mutations as compared to the general public which show less than 1% of these CFTR mutations. Regardless of the exact number in the panel, the results for each testing can be reported via report documents. For example, for those individuals that undergo testing for one or more particular SNPs disclosed herein and/or alleles/genotype at one or more SNPs, a report may be generated. The report may be a written document, such as a paper report, or reports stored in computer readable mediums, computer hard drives, computer networks, removable storage devices such as CDs, USB flash drives, or be part of databases which may optionally be accessible by the internet, or can be a report simply generated on a computer screen. The reports generated may simply include a listing of the SNPs tested for and results, such as positive or negative indicators for the SNP. Alternatively, the reports may include more information, such as a detailed analysis of the results, including significance of the positive or negative result and possible treatment modalities corresponding to the test.

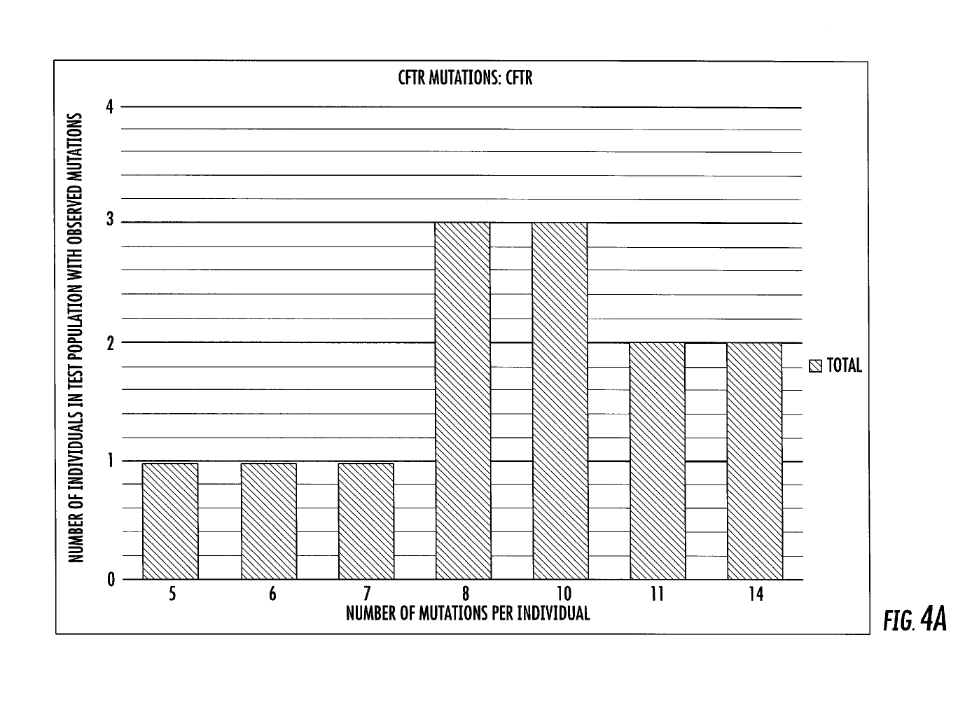

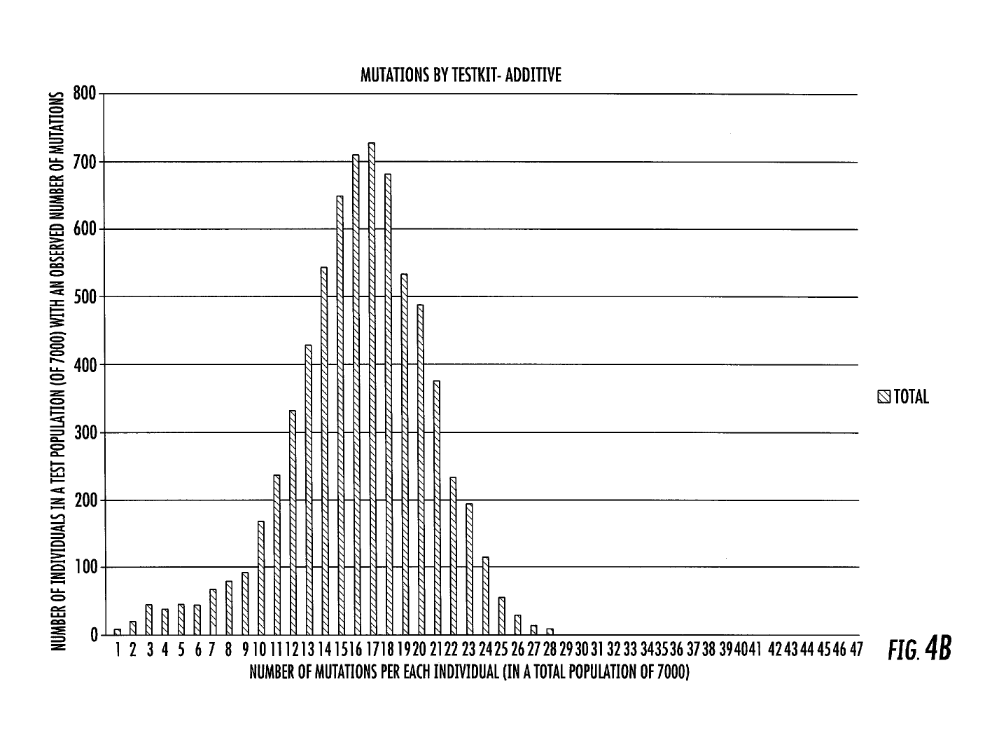

Referring to FIG. 4A, a sampling of individuals looking only at the CFTR mutations indicate that a majority of the individuals tested positive for having more than one of the of the 17 mutations tested for. As illustrated, if individuals had more than one mutation, those individuals contained between 5 and 14 mutations per individual. Referring to FIG. 4B, a group of individuals were tested and/or analyzed to determine the number of mutations per individual for all genes tested, CFTR in combination with the methylation pathway. The graph indicates a threshold number of mutations that increase the risk of autism. In looking at the composite of the SNPs for CFTR and methylation, the majority of the individuals tested had between 1 and 28 mutations per person. The highest number of mutations per person was between 12 and 21. Based on this observation and supported by for example Skafidas et al, Molecular Psychiatry 2012, 1-7 or Stone et al., Human Molecular Genetics, 2007, Vol. 16, NO 6, 704-715, one of the methods that can be employed to determine risk of developing autism is to test each individual for the CFTR related SNPs and the CFTR and methylation SNPS for the presence or absence of alleles for at each SNP position. Depending on the threshold value, which is defined as the total number of genes indicating the presence or absence of a particular SNP or overall density of number of mutations, an individual can be identified or classified as having an increased risk to developing autism. For example, an individual that contains from between 1-28, 5-25, 9-26, 10-24, or 12-22 SNPs identified out of the total number tested would be identified or classified as having an increased risk to developing autism.

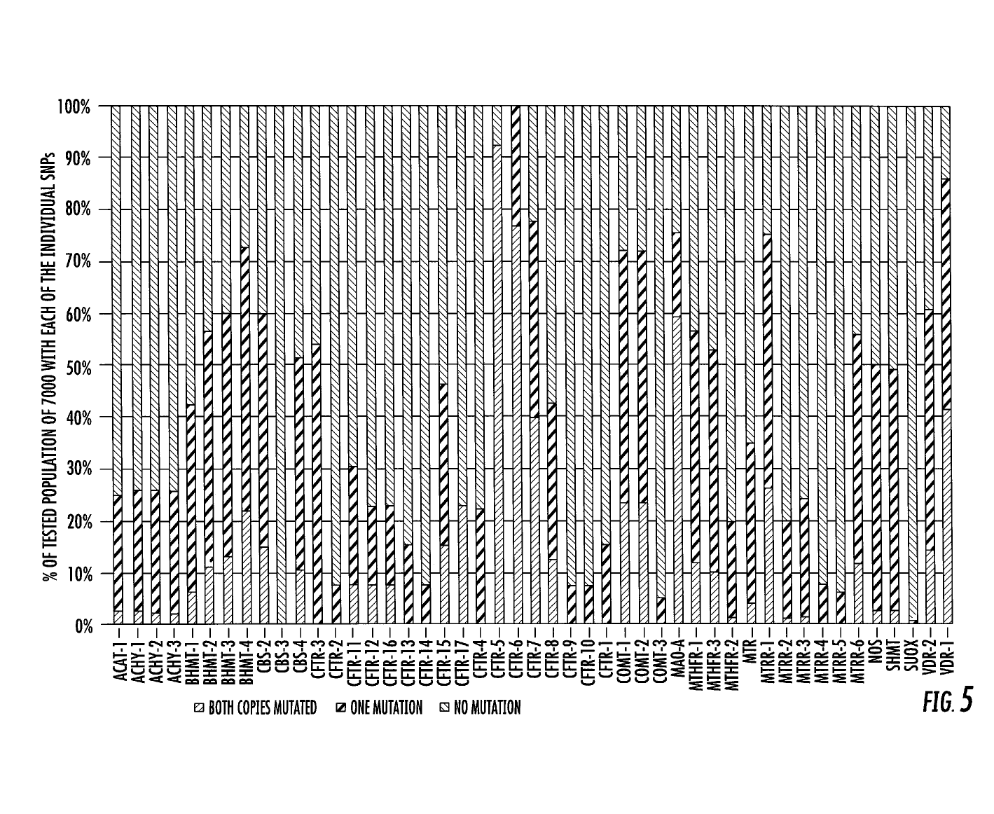

FIG. 5 illustrates the percentage of individuals in a test population which contained dual copy mutation, single copy mutation, or no mutations for both CFTR mutations and mehtylation pathway mutations. Each individual SNP is labeled on the X-axis and in coded fashion, the percentage of individuals as who had one copy of a particular SNP, two copies of a particular SNP, or no copies of the particular SNP is shown.

The present invention may also provide for isolated polynucleotide or fragments thereof comprising a genetic variation at a nucleotide position corresponding to the position of the SNP identified in FIG. 1A-1C and/or 2A-2C. The polynucleotide or fragments thereof may vary in length, ranging from 10 nucleotides in length to 1,000 nucleotides, and may include part or all naturally occurring flanking regions of the SNP.

The present invention may also provide for an oligonucleotide that is an allele specific-oligonucleotide that hybridizes to a region of a polynucleotide comprising the genetic variation at the nucleotide corresponding to the SNP location as described in FIG. 1A-1C or 2A-2C, or a complement thereof. The oligonucleotide may be used to detect the presence or absence of the variation of the polynucleotide by a method in which a nucleic acid suspected of having the variation is contacted with the oligonucleotide under suitable conditions for hybridization and detecting the presence or absence of the hybridization. The present invention may include a microarray comprising any of the oligonucleotides which are designed to detect the SNP.

The present invention may include a kit for collecting a biological sample (e.g., lancet and vial and/or absorbent card for collecting blood sample, a buccal swab for collecting buccal cells, or other sample collection device) provided to a medical practitioner, such as a physician, which the medical practitioner uses to obtain a sample (e.g., buccal cells, saliva, blood, etc.) from a patient. The sample can then be sent to a laboratory (e.g., a CLIA-certified laboratory) or other facility that tests the sample for one or more SNPs disclosed herein (e.g., to determine the genotype of one or more SNPs disclosed herein, such as to determine the patient's risk for autism), and the results of the test (e.g., the patient's genotype at one or more SNPs disclosed herein and/or the patient's disease risk based on their SNP genotype) are provided back to the medical practitioner (and/or directly to the patient and/or to another party such as a hospital, medical insurance company, genetic counselor, etc.) who may then provide or otherwise convey the results to the patient. The results are typically provided in the form of a report, such as described above.

Alternatively, kits for collecting a biological sample from a customer (e.g., a lancet and vial and/or absorbent card for collecting blood sample, a buccal swab for collecting buccal cells, or other sample collection device) are provided (e.g., for sale to a commercial business such as a drug store, pharmacy, general merchandise store, online via the internet, by mail order, etc.) whereby customers can obtain the kits, collect their own biological samples, and submit (e.g., send/deliver via mail) their samples to a laboratory or other facility which tests the samples for one or more SNPs disclosed herein (e.g., to determine the genotype of one or more SNPs disclosed herein, such as to determine the customer's risk for autism) and provides the results of the test (e.g., of the customer's genotype at one or more SNPs disclosed herein and/or the customer's disease risk based on their SNP genotype) back to the customer and/or to a third party (e.g., a physician or other medical practitioner, hospital, medical insurance company, genetic counselor, etc.). The results are typically provided in the form of a report, such as described above. If the results of the test are provided to a third party, then the third party may optionally provide another report to the customer based on the results of the test (e.g., the result of the test from the laboratory may provide the customer's genotype at one or more SNPs disclosed herein without disease risk information, and the third party may provide a report of the customer's disease risk based on this genotype result).

The SNPs of the present invention may also be used to provide SNP kits, including the detection reagents, the isolated polynucleotides as previously described. Using the known SNPs, it is possible to design SNP detection reagents that specifically detect a specific target SNP position and is specific for a particular nucleotide, or allele of the target SNP. Accordingly, the detection reagent should be designed to differentiate between alternative nucleotides at a target SNP position in order to identify the nucleotide present, thereby identifying the target SNP. For example, a probe can be used to hybridize to the nucleic acid containing the SNP by complementary base paring in sequence specific manner and discriminates the target variants sequence from other nucleic acid sequences. The probe may be designed to hybridize to the 5′ or 3′ region of a SNP position. The kit may optionally contain at least one of an enzyme, such as a nuclease, a ligase, or a polymerase. Detection methods, such as the inclusion of biotin-binding proteins, such as avidin or streptavidin, fluorescent, or radioisotope labels may be inclined as well.

The SNPs identified herein may further be used to provide targets for development of therapeutic agents for the diagnosis and treatment of genetically identified patients having autism. Individuals or subpopulations of individuals having a specific genetic signature corresponding to one or more SNPs in accordance with the present invention may utilize a targeted treatment. For example, the genes containing the genetic variations, the DNA or RNA associated with these genes, or the proteins encoded by the genes can be used as targets for the development of therapeutic agents, including small molecules compounds, antibody, or antisense technology, or be used as therapeutic agents for the treatment of autism.

Given that autism disorders are complex developmental disorders affecting brain development dealing with communication, cognition, and special interaction, and that the disorder typically manifests during the toddler years with symptoms for individuals being highly variable, a more accurate diagnostic testing modality is highly valuable. Autism may be a multi-factoral disease caused by a combination of microbial infection and environmental events which occur in genetically susceptible individuals. The present invention aims to identify those individuals who are genetically susceptible and provide autism sparing treatments. Autism sparing treatments may include simply notifying the individual and/or his/her care taker. Autism sparing treatments may include including pharmacological therapeutics, including drugs used to minimize symptoms and behaviors frequently found in individuals with autism, such as hyperactivity, impulsivity, attention difficulties, and anxiety, i.e. serotonin re-uptake inhibitors (e.g. clomipramine (Anafranil), fluvoxamine (Luvox) and fluoxetine (Prozac), anti-psychotic medications, i.e. clozapine (Clozaril), risperidone (Risperdal), olanzapine (Zyprexa) and quetiapine (Seroquel), stimulants, such as Ritalin, Adderall, and Dexedinesuch or other drugs, such as Elavil, Wellbutrin, Valium, Ativan and Xanax. Autism sparing treatments may include psychological treatments. As an alternative, Autism sparing treatments may include non-pharmacological therapeutic means of minimizing or reducing the severity of the disorder. Autism sparing treatments may include modification of lifestyle choices, such as minimizing exposure to environmental, such as toxins or vaccine timing) which may decrease the likelihood of autism. By identifying individuals susceptible to or having autism, decisions as to how to treat these individuals can be facilitated. Such decisions may be focused on non-pharmaceutical based therapeutics.

Once a person has been identified as having an increased risk of developing or diagnosed as having autism, autism sparing treatment options may be designed and utilized. Such treatments range from notification of such status to treating the individual with the standard of care of an individual with autism. Such treatment options may include one or more steps to minimize environmental factors that may play a role or otherwise contribute to the onset or severity of the disease. For example, the individual may be given one or more drugs. In an alternative embodiment, the individual may be instructed to follow non-pharmacological treatments. For instance, nutritional supplements may provide the basis for personalized genomics such that the supplements may be added to the individual's diet upon identification that the individual has one or more SNPs listed in FIG. 1, alone, or in combination with the SNPS from FIG. 2A. Except where noted, all information was obtained by the NCBI SNP database, the content of which are incorporated herein by reference

Such treatment is based on the concept that dietary chemicals can act on the human genome, directly or indirectly, to alter gene expression or structure.

In addition to nutritional supplements, the individual identified as having one or more SNPS in accordance with the recent invention may also avoid other known or autism associated factors which may play a role in the development of the disorder, such as toxins which have been associated, linked or implicated as possibly having a role in developing autism.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of determining whether an individual is predisposed to autism comprising:
obtaining a sample having genetic material from an individual;
determining the presence or absence of alleles within the CFTR gene;
classifying said individual as having an increased risk to developing autism if a predetermined number of alleles within said CFTR gene is detected.

2. The method of determining whether an individual is predisposed to autism according to claim 1 further including the step of placing said individual identified as having an increased risk to developing autism on a treatment plan.

3. The method of determining whether an individual is predisposed to autism according to claim 1 wherein said step of determining the presence or absence of alleles within the CFTR gene includes a determination of the presence or absence of an allele at rs1800073, rs1800076, rs67140043, rs1800503, rs4646205, rs10229820, rs34855237, rs213950, rs113993960, rs1800095, rs1042077, rs28517401, rs1800130, rs1800135, rs1800136, CFTR-16 (147263 C>A), CFTR-17 (Exon 19 deletion), or combinations thereof.

4. The method of determining whether an individual is predisposed to autism according to claim 1 wherein said predetermined number of alleles within the CFTR gene is between 2 and 15 independent sites within said CFTR gene.

5. The method of determining whether an individual is predisposed to autism according to claim 1 said predetermined number of alleles within the CFTR gene is a between 4 and 10 independent sites within said CFTR gene.

6. The method of determining whether an individual is predisposed to autism according to claim 1 further including the step of determining from said individual the presence or absence of alleles associated with metabolic pathway genes.

7. The method of determining whether an individual is predisposed to autism according to claim 6 wherein said a metabolic pathway includes genes associated with the methylation pathway.

8. The method of determining whether an individual is predisposed to autism according to claim 7 wherein said methylation genes includes a determination of the presence or absence of an allele at rs4633, rs4680, rs769224, rs731236, rs2228570, rs6323, rs3741049, rs1801133, rs2066470, rs1801131, rs1805087, rs1801394, rs10380, rs162036, rs2287780, rs2303080, rs1802059, rs585800, rs567754, rs617219, rs651852, rs819147, rs819134, rs819171, rs234706, rs1801181, rs2298758, rs773115, rs197927, rs1799983, rs12325817, rs7946, or combinations thereof.

9. The method of determining whether an individual is predisposed to autism according to claim 8 further including the step of placing said individual identified as having a predisposition to autism on a treatment plan.

10. A method of determining whether an individual has an altered risk for developing autism comprising the steps of:
obtaining a sample having genetic material from an individual;
determining the presence or absence of SNP at rs1800073, rs1800076, rs67140043, rs1800503, rs4646205, rs10229820, rs34855237, rs213950, rs113993960, rs1800095, rs1042077, rs28517401, rs1800130, rs1800135, rs1800136, CFTR-16 (147263 C>A), CFTR-17 (Exon 19 deletion);
determining the presence or absence of a SNP at rs4633, rs4680, rs769224, rs731236, rs2228570, rs6323, rs3741049, rs1801133, rs2066470, rs1801131, rs1805087, rs1801394, rs10380, rs162036, rs2287780, rs2303080, rs1802059, rs585800, rs567754, rs617219, rs651852, rs819147, rs819134, rs819171, rs234706, rs1801181, rs2298758, rs773115, rs197927, rs1799983, rs12325817, rs7946;
identifying said individual as having an altered risk of developing autism if said human contains a threshold value of SNPs.

11. The method of determining whether an individual is predisposed to autism according to claim 10 wherein said threshold value is the presence or absence of between 1 and 28 SNPs.

12. The method of determining whether an individual is predisposed to autism according to claim 10 wherein said threshold value is the presence or absence of between 5 and 25 SNPs.

13. The method of determining whether an individual is predisposed to autism according to claim 10 wherein said threshold value is the presence or absence of between 10 and 24 SNPs.

14. The method of determining whether an individual is predisposed to autism according to claim 10 wherein said threshold value is the presence or absence of between 12 and 22 SNPs.

15. The method of determining whether an individual has an altered risk for developing autism according to claim 10 wherein said SNPs are homozygous or heterozygous.

16. The method of determining whether an individual has an altered risk for developing autism according to claim 10 wherein said nucleic acid is extracted from a biological sample from said individual.

17. The method of determining whether an individual has an altered risk for developing autism according to claim 16 wherein said biological sample is blood, saliva, mucosal scraping, or tissue biopsy.

18. The method of determining whether an individual has an altered risk for developing autism according to claim 10 further including the step of treating said individual identified as having an altered risk for developing autism with autism sparing treatments.

19. The method of determining whether an individual has an altered risk for developing autism according to claim 18 wherein said autism sparing treatments includes minimizing said individual from engaging with one or more factors linked to development of autism.

20. The method of determining whether an individual has an altered risk for developing autism according to claim 18 wherein said autism sparing treatments includes nutritional supplements.

21. A set of SNPs comprising a genetic signature indicative of the risk of developing autism, wherein said set of SNPs comprises rs1800073, rs1800076, rs67140043, rs1800503, rs4646205, rs10229820, rs34855237, rs213950, rs113993960, rs1800095, rs1042077, rs28517401, rs1800130, rs1800135, rs1800136, CFTR-16 (147263 C>A), CFTR-17 (Exon 19 deletion), or combinations thereof.

22. The set of SNPs comprising a genetic signature indicative of the risk of developing autism according to claim 21 further including one or more SNPs selected from rs4633, rs4680, rs769224, rs731236, rs2228570, rs6323, rs3741049, rs1801133, rs2066470, rs1801131, rs1805087, rs1801394, rs10380, rs162036, rs2287780, rs2303080, rs1802059, rs585800, rs567754, rs617219, rs651852, rs819147, rs819134, rs819171, rs234706, rs1801181, rs2298758, rs773115, rs197927, rs1799983, rs12325817, rs7946, or combination thereof.


SEQUENCE LISTING (50 sequences)

SEQ ID NO: 1 - DNA, 52 bp, Homo sapiens
caattttgaggaaaggatacagacagygcctggaattgtcagacatatacca

SEQ ID NO: 2 - DNA, 52 bp, Homo sapiens
tcctaaactcattaatgcccttcggcratgttttttctggagatttatgttc

SEQ ID NO: 3 - DNA, 51 bp, Homo sapiens
  misc_feature (27)..(30): This region may or may not be present in its entirety
gataatttgacttgtttttactattagattgattgattgattgattgattt

SEQ ID NO: 4 - DNA, 52 bp, Homo sapiens
ttgaaaacttaagacagtaagttgttycaataatttcaatattgttagtaat

SEQ ID NO: 5 - DNA, 53 bp, Homo sapiens
  misc_feature (27)..(28): This region may or may not be present in its entirety
tgatgtgtgtgtgtgtgtgtgtgtgtgtttttttaacagggatttggggaatt

SEQ ID NO: 6 - DNA, 52 bp, Homo sapiens
tttgatgtgtgtgtgtgtgtgtgtgtktttttttaacagggatttggggaat

SEQ ID NO: 7 - DNA, 52 bp, Homo sapiens
acacttctgcttaggatgataattggrggcaagtgaatcctgagcgtgattt

SEQ ID NO: 8 - DNA, 52 bp, Homo sapiens
tttatttccagacttcacttctaatgrtgattatgggagaactggagccttc

SEQ ID NO: 9 - DNA, 54 bp, Homo sapiens
  misc_feature (27)..(29): This region may or may not be present in its entirety
cctggcaccattaaagaaaatatcatctttggtgtttcctatgatgaatataga

SEQ ID NO: 10 - DNA, 52 bp, Homo sapiens
gtcatcaaagcatgccaactagaagargtaagaaactatgtgaaaacttttt

SEQ ID NO: 11 - DNA, 52 bp, Homo sapiens
tggaacacataccttcgatatattackgtccacaagagcttaatttttgtgc

SEQ ID NO: 12 - DNA, 52 bp, Homo sapiens
tgcgatactcatcttgtaaaaaagctwtaagagctatttgagattctttatt

SEQ ID NO: 13 - DNA, 52 bp, Homo sapiens
tggaggaaagcctttggagtgataccrcaggtgagcaaaaggacttagccag

SEQ ID NO: 14 - DNA, 52 bp, Homo sapiens
atagaagagaacaaagtgcggcagtaygattccatccagaaactgctgaacg

SEQ ID NO: 15 - DNA, 52 bp, Homo sapiens
tcaagcaagtgcaagtctaagccccarattgctgctctgaaagaggagacag

SEQ ID NO: 16 - DNA, 21 bp, Homo sapiens
ttttaggaagmatcaaactaa

SEQ ID NO: 17 - DNA, 52 bp, Homo sapiens
accaaggagcagcgcatcctgaaccaygtgctgcagcatgcggagcccggga

SEQ ID NO: 18 - DNA, 52 bp, Homo sapiens
cccagcggatggtggatttcgctggcrtgaaggacaaggtgtgcatgcctga

SEQ ID NO: 19 - DNA, 52 bp, Homo sapiens
gaccactggaaggaccggtacctgccrgacacgcttctcttggaggtgagcc

SEQ ID NO: 20 - DNA, 52 bp, Homo sapiens
cctggggtgcaggacgccgcgctgatygaggccatccaggaccgcctgtcca

SEQ ID NO: 21 - DNA, 52 bp, Homo sapiens
  modified_base (27)..(27): a, c, t or g
tggcctgcttgctgttcttacaggganggaggcaatggcggccagcacttcc

SEQ ID NO: 22 - DNA, 52 bp, Homo sapiens
gcagagagaaaccagttaattcagcgkcttccaatgggagctgtcattaagt

SEQ ID NO: 23 - DNA, 52 bp, Homo sapiens
aaatttctttaatgactgagccatgcygatgcaataggcatacaaaaatatt

SEQ ID NO: 24 - DNA, 52 bp, Homo sapiens
cttgaaggagaaggtgtctgcgggagycgatttcatcatcacgcagcttttc

SEQ ID NO: 25 - DNA, 52 bp, Homo sapiens
agatgttccaccccgggcctggacccygagcggcatgagagactccgggaga

SEQ ID NO: 26 - DNA, 52 bp, Homo sapiens
gtggggggaggagctgaccagtgaagmaagtgtctttgaagtcttcgttctt

SEQ ID NO: 27 - DNA, 52 bp, Homo sapiens
ggaagaatatgaagatattagacaggrccattatgagtctctcaaggtaagt

SEQ ID NO: 28 - DNA, 52 bp, Homo sapiens
caggcaaaggccatcgcagaagaaatrtgtgagcaagctgtggtacatggat

SEQ ID NO: 29 - DNA, 52 bp, Homo sapiens
tattatatttcagaaaagagctcagayatttccttaagcatgggatcttaac

SEQ ID NO: 30 - DNA, 52 bp, Homo sapiens
ccttttgaaaataaaggcagacacaargaagaaaggtaacagccctgatgct

SEQ ID NO: 31 - DNA, 52 bp, Homo sapiens
gtaaacaaggggcagccgattatagcygctttgtacgagatgcctgtgcctg

SEQ ID NO: 32 - DNA, 52 bp, Homo sapiens
aatatttacaggtacatctgcaggagwctcttggccaggtaaggaagttttt

SEQ ID NO: 33 - DNA, 52 bp, Homo sapiens
atccagcttcatggccagcaggtggcragaatcctcctccaggagaacggcc

SEQ ID NO: 34 - DNA, 52 bp, Homo sapiens
gtgaaaagtattatggaaatcactgcwgcacaggaaaagtaattcagatgtt

SEQ ID NO: 35 - DNA, 52 bp, Homo sapiens
agacaaatacacctagtacattttctytaccttttgctttcaagagtactgt

SEQ ID NO: 36 - DNA, 52 bp, Homo sapiens
acccctaaaggaaacaagaactgcgcmggaatgaatcaagaacatccgttcc

SEQ ID NO: 37 - DNA, 52 bp, Homo sapiens
ttcatttagtacatttagggactggcraaattctcaccctcctttgactggt

SEQ ID NO: 38 - DNA, 52 bp, Homo sapiens
tgctctatataacgggattacgtccayctctatgaagattattgtgtaaaca

SEQ ID NO: 39 - DNA, 52 bp, Homo sapiens
acagccaatatttattaagaaatgttragctggccggggcacggtggctcac

SEQ ID NO: 40 - DNA, 52 bp, Homo sapiens
gacagccgttccactgcagagtacccygtgaatgcttcatcagcacaactat

SEQ ID NO: 41 - DNA, 52 bp, Homo sapiens
tgcaggatctcatcagcggtggtgtcrtagtgagccagggggttgctggcgt

SEQ ID NO: 42 - DNA, 52 bp, Homo sapiens
agcacggtggcggtggccgtgaaggcygcgcaggagctgcaggagggccagc

SEQ ID NO: 43 - DNA, 52 bp, Homo sapiens
tctaggatgtgagaattggggatttcrttcttcagccgccaggccaccccca

SEQ ID NO: 44 - DNA, 52 bp, Homo sapiens
tctccatcccggggctctgtgatggcvgactggacaggaagttcctgaatgg

SEQ ID NO: 45 - DNA, 52 bp, Homo sapiens
cccacagccaattcccgggccatgcargccggaatgcacggggtcattagcg

SEQ ID NO: 46 - DNA, 52 bp, Homo sapiens
cccctgctgctgcaggccccagatgakccccagaactcttccttctgccccc

SEQ ID NO: 47 - DNA, 52 bp, Homo sapiens
ccagcctggacaacatggtgacactcsgtctctactaaaaatacaaaaatag

SEQ ID NO: 48 - DNA, 52 bp, Homo sapiens
tactcactcttcgtataggagagccaytatgtaggtgagggccaccagcacc

SEQ ID NO: 49 - DNA, 93 bp, Homo sapiens
gatttcttacaaaagcaagaatataagacattggaatataacttaacgactacagaagtagtgatggagaatgtaacagccttctgggaggag

SEQ ID NO: 50 - DNA, 22 bp, Artificial Sequence
  Description of Artificial Sequence Synthetic primer
tgctttgatgacgcttctgtat